(19) United States
(12) Patent Application Publication
(10) Pub. No.: US 2009/0176257 A1
(43) Pub. Date: Jul. 9, 2009

(54) Methods and Biomarkers for Diagnosing and Monitoring Psychotic Disorders

(76) Inventors: Sabine Bahn, Cambridge (GB); Jeffery T. -J Huang, Cambridge (GB); Tsz Tsang, London (GB)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/090,530

(22) PCT Filed: Oct. 18, 2006
(86) PCT No.: PCT/GB2006/003870

§ 371 Date: Feb. 22, 2009

(30) Foreign Application Priority Data
Oct. 18, 2005 (GB) 0521098.4
Dec. 30, 2005 (GB) 0526557.4
Apr. 6, 2006 (GB) 0606920.7

(51) Int. Cl. G01N 33/68; G01N 24/08; G01N 24/00

(52) U.S. Cl. 435/7.92; 436/173

(57) ABSTRACT

The invention relates to methods of diagnosing or monitoring a psychotic disorder in a subject comprising providing a test biological sample from the subject, performing spectral analysis on said test biological sample to provide one or more spectra, and, comparing the one or more spectra with one or more control spectra. The invention also relates to methods for diagnosing or monitoring psychotic disorders such as schizophrenic or bipolar disorders, comprising measuring the level of one or more biomarkers present in a biological sample taken from a test subject, said biomarkers being selected from the group consisting of transthyretin, ApoA1: VLDL, LDL and aromatic species such as plasma proteins. The invention also relates to sensors, biosensors, multi-analyte panels, arrays, assays and kits for performing methods of the invention.

### TECHNICAL FIELD

The present invention relates to methods of diagnosing or of monitoring psychotic disorders, in particular schizophrenic disorders (and bipolar disorders), e.g. using biomarkers. The biomarkers and methods in which they are employed can be used to assist diagnosis and to assess onset and development of psychotic disorders. The invention also relates to use of biomarkers in clinical screening, assessment of prognosis, evaluation of therapy, for drug screening and drug development.

### BACKGROUND OF THE INVENTION

Psychosis is a symptom of severe mental illness. Although it is not exclusively linked to any particular psychological or physical state, it is particularly associated with schizophrenia, bipolar disorder (manic depression) and severe clinical depression. Psychosis is characterized by disorders in basic perceptual, cognitive, affective and judgmental processes. Individuals experiencing a psychotic episode may experience hallucinations (often auditory or visual hallucinations), hold paranoid or delusional beliefs, experience personality changes and exhibit disorganised thinking (thought disorder). This is sometimes accompanied by features such as a lack of insight into the unusual or bizarre nature of their behaviour, difficulties with social interaction and impairments in carrying out the activities of daily living.

Psychosis is not uncommon in cases of brain injury and may occur after drug use, particularly after drug overdose or chronic use; certain compounds may be more likely to induce psychosis and some individuals may show greater sensitivity than others. The direct effects of hallucinogenic drugs are not usually classified as psychosis, as long as they abate when the drug is metabolised from the body. Chronic psychological stress is also known to precipitate psychotic states, however the exact mechanism is uncertain. Psychosis triggered by stress in the absence of any other mental illness is known as brief reactive psychosis. Psychosis is thus a descriptive term for a complex group of behaviours and experiences. Individuals with schizophrenia can have long periods without psychosis and those with bipolar disorder, or depression, can have mood symptoms without psychosis.

Hallucinations are defined as sensory perception in the absence of external stimuli. Psychotic hallucinations may occur in any of the five senses and can take on almost any form, which may include simple sensations (such as lights, colours, tastes, smells) to more meaningful experiences such as seeing and interacting with fully formed animals and people, hearing voices and complex tactile sensations. Auditory hallucination, particularly the experience of hearing voices, is a common and often prominent feature of psychosis. Hallucinated voices may talk about, or to the person, and may involve several speakers with distinct personas. Auditory hallucinations tend to be particularly distressing when they are derogatory, commanding or preoccupying.

Psychosis may involve delusional or paranoid beliefs, classified into primary and secondary types. Primary delusions are defined as arising out-of-the-blue and not being comprehensible in terms of normal mental processes, whereas secondary delusions may be understood as being influenced by the person's background or current situation, i.e. represent a delusional interpretation of a “real” situation.

Thought disorder describes an underlying disturbance to conscious thought and is classified largely by its effects on the content and form of speech and writing. Affected persons may also show pressure of speech (speaking incessantly and quickly), derailment or flight of ideas (switching topic mid-sentence or inappropriately), thought blocking, rhyming or punning.

Psychotic episodes may vary in duration between individuals. In brief reactive psychosis, the psychotic episode is commonly related directly to a specific stressful life event, so patients spontaneously recover normal functioning, usually within two weeks. In some rare cases, individuals may remain in a state of full blown psychosis for many years, or perhaps have attenuated psychotic symptoms (such as low intensity hallucinations) present at most times.

Patients who suffer a brief psychotic episode may have many of the same symptoms as a person who is psychotic as a result of, for example, schizophrenia, and this fact has been used to support the notion that psychosis is primarily a breakdown in some specific biological system in the brain.

Schizophrenia is a major psychotic disorder affecting up to 1% of the population. It is found at similar prevalence in both sexes and is found throughout diverse cultures and geographic areas. The World Health Organization found schizophrenia to be the world's fourth leading cause of disability that accounts for 1.1% of the total DALYs (Disability Adjusted Life Years) and 2.8% of YLDs (years of life lived with disability). It was estimated that the economic cost of schizophrenia exceeded US$ 19 billion in 1991, more than the total cost of all cancers in the United States. Early diagnosis and effective treatment of schizophrenia can improve prognosis and help reduce the costs associated with this illness.

The clinical syndrome of schizophrenia comprises discrete clinical features including positive symptoms (hallucination, delusions, disorganization of thought and bizarre behaviour); negative symptoms (loss of motivation, restricted range of emotional experience and expression and reduced hedonic capacity); and cognitive impairments with extensive variation between individuals. No single symptom is unique to schizophrenia and/or is present in every case. Despite the lack of homogeneity of clinical symptoms, the current diagnosis and classification of schizophrenia is still based on the clinical symptoms presented by a patient. This is primarily because the aetiology of schizophrenia remains unknown (in fact, the aetiology of most psychiatric diseases is still unclear) and classification based on aetiology is as yet not feasible. The clinical symptoms of schizophrenia are often similar to symptoms observed in other neuropsychiatric and neurodevelopmental disorders.

Due to the complex spectrum of symptoms presented by subjects with schizophrenic disorders and their similarity to other mental disorders, current diagnosis of schizophrenia is made on the basis of a complicated clinical examination/interview of the patient's family history, personal history, current symptoms (mental state examination) and the presence/absence of other disorders (differential diagnosis). This assessment allows a “most likely” diagnosis to be established, leading to the initial treatment plan. To be diagnosed with schizophrenia, a patient (with few exceptions) should have psychotic, “loss-of-reality” symptoms for at least six months (DSM IV) and show increasing difficulty in functioning normally.

The ICD-10 Classification of Mental and Behavioural Disorders, published by the World Health Organization in 1992, is the manual most commonly used by European psychiatrists to diagnose mental health conditions. The manual provides detailed diagnostic guidelines and defines the various forms of schizophrenia: schizophrenia, paranoid schizophrenia, hebrephrenic schizophrenia, catatonic schizophrenia, undifferentiated schizophrenia, post-schizophrenic schizophrenia, residual schizophrenia and simple schizophrenia.

The Diagnostic and Statistical Manual of Mental Disorders fourth edition (DSM IV) published by the American Psychiatric Association, Washington D.C., 1994, has proven to be an authoritative reference handbook for health professionals both in the United Kingdom and in the United States in categorising and diagnosing mental health problems. This describes the diagnostic criteria, subtypes, associated features and criteria for differential diagnosis of mental health disorders, including schizophrenia, bipolar disorder and related psychotic disorders.

### DSM IV Diagnostic Criteria for Schizophrenia

A. Characteristic symptoms: Two (or more) of the following, each present for a significant portion of time during a 1-month period (or less if successfully treated): delusions, hallucinations, disorganized speech (e.g., frequent derailment or incoherence), grossly disorganized or catatonic behaviour, negative symptoms, i.e., affective flattening, alogia, or avolition. Only one Criterion A symptom is required if delusions are bizarre or hallucinations consist of a voice keeping up a running commentary on the person's behaviour or thoughts, or two or more voices conversing with each other.
B. Social/occupational dysfunction: For a significant portion of the time since the onset of the disturbance, one or more major areas of functioning such as work, interpersonal relations, or self-care are markedly below the level achieved prior to the onset (or when the onset is in childhood or adolescence, failure to achieve expected level of interpersonal, academic, or occupational achievement).
C. Duration: Continuous signs of the disturbance persist for at least 6 months. This 6-month period must include at least 1 month of symptoms (or less if successfully treated) that meet Criterion A (i.e., active-phase symptoms) and may include periods of prodromal or residual symptoms. During these prodromal or residual periods, the signs of the disturbance may be manifested by only negative symptoms or two or more symptoms listed in Criterion A present in an attenuated form (e.g., odd beliefs, unusual perceptual experiences).
D. Schizoaffective and Mood Disorder exclusion: Schizoaffective Disorder and Mood Disorder With Psychotic Features have been ruled out because either (1) no Major Depressive Episode, Manic Episode, or Mixed Episode have occurred concurrently with the active-phase symptoms; or (2) if mood episodes have occurred during active-phase symptoms, their total duration has been brief relative to the duration of the active and residual periods.
E. Substance/general medical condition exclusion: The disturbance is not due to the direct physiological effects of a substance (e.g., a drug of abuse, a medication) or a general medical condition, so-called “organic” brain disorders/syndromes.
F. Relationship to a Pervasive Developmental Disorder: If there is a history of Autistic Disorder or another Pervasive Developmental Disorder, the additional diagnosis of Schizophrenia is made only if prominent delusions or hallucinations are also present for at least a month (or less if successfully treated).

### Schizophrenia Subtypes

1. Paranoid Type: A type of Schizophrenia in which the following criteria are met: preoccupation with one or more delusions (especially with persecutory content) or frequent auditory hallucinations. None of the following is prominent: disorganized speech, disorganized or catatonic behaviour, or flat or inappropriate affect.
2. Catatonic Type: A type of Schizophrenia in which the clinical picture is dominated by at least two of the following: motoric immobility as evidenced by catalepsy (including waxy flexibility) or stupor excessive motor activity (that is apparently purposeless and not influenced by external stimuli), extreme negativism (an apparently motiveless resistance to all instructions or maintenance of a rigid posture against attempts to be moved) or mutism, peculiarities of voluntary movement as evidenced by posturing (voluntary assumption of inappropriate or bizarre postures), stereotyped movements, prominent mannerisms, or prominent grimacing echolalia or echopraxia.
3. Disorganized Type: A type of Schizophrenia in which the following criteria are met: all of the following are prominent: disorganized speech, disorganized behaviour, flat or inappropriate affect. The criteria are not met for the Catatonic Type.
4. Undifferentiated Type: A type of Schizophrenia in which symptoms that meet Criterion A are present, but the criteria are not met for the Paranoid, Disorganized, or Catatonic Type.
5. Residual Type: A type of Schizophrenia in which the following criteria are met: absence of prominent delusions, hallucinations, disorganized speech, and grossly disorganized or catatonic behaviour. There is continuing evidence of the disturbance, as indicated by the presence of negative symptoms or two or more symptoms listed in Criterion A for Schizophrenia, present in an attenuated form (e.g., odd beliefs, unusual perceptual experiences).

### Schizophrenia Associated Features

Features associated with schizophrenia include: learning problems, hypoactivity, psychosis, euphoric mood, depressed mood, somatic or sexual dysfunction, hyperactivity, guilt or obsession, sexually deviant behaviour, odd/eccentric or suspicious personality, anxious or fearful or dependent personality, dramatic or erratic or antisocial personality.

Many disorders have similar or even the same symptoms as schizophrenia: psychotic disorder due to a general medical condition, delirium, or dementia; substance-induced psychotic disorder; substance-induced delirium; substance-induced persisting dementia; substance-related disorders; mood disorder with psychotic features; schizoaffective disorder; depressive disorder not otherwise specified; bipolar disorder not otherwise specified; mood disorder with catatonic features; schizophreniform disorder; brief psychotic disorder; delusional disorder; psychotic disorder not otherwise specified; pervasive developmental disorders (e.g., autistic disorder); childhood presentations combining disorganized speech (from a communication disorder) and disorganized behaviour (from attention-deficit/hyperactivity disorder); schizotypal disorder; schizoid personality disorder and paranoid personality disorder.

### DSM IV Diagnostic Categories for Manic Depression/Bi-Polar Affective Disorder (BD)

Only two sub-types of bipolar illness have been defined clearly enough to be given their own DSM categories, Bipolar I and Bipolar II.

Bipolar I: This disorder is characterized by manic episodes; the ‘high’ of the manic-depressive cycle. Generally this manic period is followed by a period of depression, although some bipolar I individuals may not experience a major depressive episode. Mixed states, where both manic or hypomanic symptoms and depressive symptoms occur at the same time, also occur frequently with bipolar I patients (for example, depression with the racing thoughts of mania). Also, dysphoric mania is common, this is mania characterized by anger and irritability.
Bipolar II: This disorder is characterized by major depressive episodes alternating with episodes of hypomania, a milder form of mania. Hypomanic episodes can be a less disruptive form of mania and may be characterized by low-level, non-psychotic symptoms of mania, such as increased energy or a more elated mood than usual. It may not affect an individual's ability to function on a day to day basis. The criteria for hypomania differ from those for mania only by their shorter duration (at least 4 days instead of 1 week) and milder severity (no marked impairment of functioning, hospitalization or psychotic features).

If alternating episodes of depressive and manic symptoms last for two years and do not meet the criteria for a major depressive or a manic episode then the diagnosis is classified as a Cyclothymic disorder, which is a less severe form of bipolar affective disorder. Cyclothymic disorder is diagnosed over the course of two years and is characterized by frequent short periods of hypomania and depressive symptoms separated by periods of stability.

Rapid cycling occurs when an individual's mood fluctuates from depression to hypomania or mania in rapid succession with little or no periods of stability in between. One is said to experience rapid cycling when one has had four or more episodes, in a given year, that meet criteria for major depressive, manic, mixed or hypomanic episodes. Some people who rapid cycle can experience monthly, weekly or even daily shifts in polarity (sometimes called ultra rapid cycling).

When symptoms of mania, depression, mixed mood, or hypomania are caused directly by a medical disorder, such as thyroid disease or a stroke, the current diagnosis is Mood Disorder Due to a General Medical Condition.

If a manic mood is brought about through an antidepressant, ECT or through an individual using street drugs, the diagnosis is Substance-induced Mood Disorder, with Manic Features.

Diagnosis of Bipolar III has been used to categorise manic episodes which occur as a result of taking an antidepressant medication, rather than occurring spontaneously. Confusingly, it has also been used in instances where an individual experiences hypomania or cyclothymia (i.e. less severe mania) without major depression.

### Mania

Manic Depression is comprised of two distinct and opposite states of mood, whereby depression alternates with mania. The DSM IV gives a number of criteria that must be met before a disorder is classified as mania. The first one is that an individual's mood must be elevated, expansive or irritable. The mood must be a different one to the individual's usual affective state during a period of stability. There must be a marked change over a significant period of time. The person must become very elevated and have grandiose ideas. They may also become very irritated and may well appear to be ‘arrogant’ in manner. The second main criterion for mania emphasizes that at least three of the following symptoms must have been present to a significant degree: inflated sense of self importance, decreased need for sleep, increased talkativeness, flight of ideas or racing thoughts, easily distracted, increased goal-directed activity. Excessive involvement in activities that can bring pleasure but may have disastrous consequences (e.g. sexual affairs and spending excessively). The third criterion for mania in the DSM IV emphasizes that the change in mood must be marked enough to affect an individual's job performance or ability to take part in regular social activities or relationships with others. This third criterion is used to emphasize the difference between mania and hypomania.

### Depression

The DSM IV states that there are a number of criteria by which major depression is clinically defined. The condition must have been evident for at least two weeks and must have five of the following symptoms: a depressed mood for most of the day, almost every day, a loss of interest or pleasure in almost all activities, almost every day, changes in weight and appetite, sleep disturbance, a decrease in physical activity, fatigue and loss of energy, feelings of worthlessness or excessive feelings of guilt, poor concentration levels, suicidal thoughts.

Both the depressed mood and a loss of interest in everyday activities must be evident as two of the five symptoms which characterize a major depression. It is difficult to distinguish the symptoms of an individual suffering from the depressed mood of manic depression from someone suffering from a major depression. Dysthymia is a less severe depression than unipolar depression, but it can be more persistent.

The prolonged process currently needed to achieve accurate diagnosis of psychotic disorders may delay appropriate treatment, which is likely to have serious implications for medium to long-term disease outcome. The development of objective diagnostic methods, tests and tools is urgently required to help distinguish between psychiatric diseases with similar clinical symptoms. Objective diagnostic methods and tests for psychotic disorders, such as schizophrenia and/or bipolar disorder, will assist in monitoring individuals over the course of illness (treatment response, compliance etc.) and may also be useful in determining prognosis, as well as providing tools for drug screening and drug development.

Unfortunately, at present there are no standard, sensitive, specific tests for psychotic disorders, such as schizophrenia or bipolar disorders.

One biochemical test currently under development for schizophrenia diagnosis is the niacin skin flush test, based on the observation that there is failure to respond to the niacin skin test in some schizophrenia patients, due to abnormal arachidonic acid metabolism. However, the specificity and sensitivity of this test shows an extreme inconsistency between studies, ranging from 23% to 87%, suggesting that the reliability and validity of this test still need to be verified.

WO 01/63295 describes methods and compositions for screening, diagnosis, and determining prognosis of neuropsychiatric or neurological conditions, including BAD (bipolar affective disorder), schizophrenia and vascular dementia, for monitoring the effectiveness of treatment in these conditions and for use in drug development.

Other techniques such as magnetic resonance imaging or positron emission tomography, based on subtle changes of the frontal and temporal lobes and the basal ganglia are of little value for the diagnosis, treatment, or prognosis of schizophrenic disorders in individual patients, since the absolute size of these reported differences between individuals with schizophrenia and normal comparison subjects has been generally small, with notable overlap between the two groups. The role of these neuroimaging techniques is restricted largely to the exclusion of other conditions which may be accompanied by schizophrenic symptoms, such as brain tumours or haemorrhages.

Metabonomic studies can be used to generate a characteristic pattern or “fingerprint” of the metabolic status of an individual. Metabonomic studies on biofluids provide information on the biochemical status of the whole organism, since the composition of a given biofluid is a consequence of the function of the cells that are intimately concerned with the fluid's composition and secretion.

“Metabonomics” is conventionally defined as “the quantitative measurement of the multi-parametric metabolic response of living systems to pathophysiological stimuli or genetic modification”. Metabonomics has developed from the use of ¹H NMR spectroscopy to study the metabolic composition of biofluids, cells, and tissues and from studies utilising pattern recognition (PR), expert systems and other chemoinformatic tools to interpret and classify complex NMR-generated metabolic data sets and to extract useful biological information.

¹H NMR spectra of biofluids and tissues provide a characteristic metabolic “fingerprint” or profile of the organism from which the biofluid was obtained for a range of biologically-important endogenous metabolites. This metabolic profile is characteristically changed by a disease, disorder, toxic process, or xenobiotic (e.g. drug substance). Quantifiable differences in metabolite patterns in biofluids and tissues can give information and insight into the underlying molecular mechanisms of disease or disorder. In the evaluation of the effects of drugs, each compound or class of compound produces characteristic changes in the concentrations and patterns of endogenous metabolites in biofluids.

The metabolic changes can be characterised using automated computer programs which represent each metabolite measured in the biofluid spectrum as a co-ordinate in multi-dimensional space.

Metabonomic technology has been used to identify biomarkers of inborn errors of metabolism, liver and kidney disease, cardiovascular disease, insulin resistance and neurodegenerative disorders.

The current diagnosis of psychotic disorders, such as schizophrenia, remains subjective, not only because of the complex spectrum of symptoms and their similarity to other mental disorders, but also due to the lack of empirical disease markers. There is a great clinical need for diagnostic tests and more effective drugs to treat severe mental illnesses.

Recent functional genomics studies suggest that there may be a metabolic component to the schizophrenia syndrome, but the contribution of metabolic aspects to psychotic disease is poorly understood. There is some evidence that abnormal glucoregulation, lipid metabolism and mitochondrial dysfunction are associated with schizophrenia and affective disorders⁸⁻¹¹. But it is not understood if these metabolic alterations are a cause or effect of the disorder itself, or whether they occur as a result of medication. Antipsychotic drug action has been prominently linked to dyslipidemia, but reports of altered glucose metabolism predate the antipsychotic era (reviewed by Haupt and Newcomer¹²) and a recent report aimed at determining the rate of metabolic syndrome in long-term schizophrenia patients found the prevalence of metabolic syndrome to be inversely correlated to the daily dose of antipsychotic drugs¹³.

It is now widely accepted that both genetic and non-genetic environmental factors contribute to the aetiopathology of schizophrenia and/or precipitate the onset of the syndrome. Numerous biological (viral exposure³, illicit drug use⁴, perinatal insults⁵ etc.) and social stressors are considered to be environmental disease components, likely to interact with a predisposing genotype. Twin studies are particularly powerful tools for unraveling genetic and environmental factors responsible for complex disorders. Previous studies have demonstrated that the likelihood to develop schizophrenia correlates highly with the level of consanguinity and reaches a concordance rate of about 30-50% for monozygotic twins^{1,2}. Investigations of discordant twins, i.e. twins in which one twin presents with a disorder and the other twin does not, may help to disentangle the impact of some of these components. Due to the difficulties in obtaining brain samples from discordant twins in sufficient numbers, studies of monozygotic twins discordant for schizophrenia have so far focused on brain imaging. Twin studies imply that one of the most consistently reported brain alterations in schizophrenia, i.e. lateral ventricular enlargement, can been attributed to environmental factors^{6,7}

Biomarkers present in readily accessible body fluids, such as blood, plasma, serum, urine, saliva or cerebrospinal fluid (CSF), may prove useful in diagnosis of psychotic disorders, aid in predicting and monitoring treatment response and compliance, and assist in identification of novel drug targets. Appropriate biomarkers are also important tools in development of new early or pre-symptomatic treatments designed to improve outcomes or to prevent pathology.

The validation of biomarkers that can detect early changes specifically correlated to reversal or progression of mental disorders is essential for monitoring and optimising interventions. Used as predictors, these biomarkers can help to identify high-risk individuals and disease sub-groups that may serve as target populations for chemo-intervention trials; whilst as surrogate endpoints, biomarkers have the potential for assessing the efficacy and cost effectiveness of preventative interventions at a speed which is not possible at present when the incidence of manifest mental disorder is used as the endpoint.

The transthyretin gene encodes a plasma protein transthyretin (TTR) that belongs to a group of proteins, including thyroxine-binding globulin and albumin, which bind and transport thyroid hormones in the blood. TTR transports thyroxine from the bloodstream to the brain¹⁵. It is a single polypeptide chain of 127 amino acids (14 kDa) and is present in the plasma as a tetramer of non-covalently bound monomers. TTR is expressed at a high rate in the brain choroid plexus, from which it is released into the cerebrospinal fluid (CSF). In peripheral tissues, it is expressed primarily in liver. Only an estimated 3% of transthyretin in the ventricular CSF and only 10% of the transthyretin in lumbar CSF are derived from blood¹⁶. Under physiological conditions, the macromolecular complex plays an important physiological role in vitamin A homeostasis because it binds the specific transport protein for retinol, the lipocalin retinol-binding protein (RBP). This reduces the glomerular filtration of the low molecular weight transport protein (21 kDa) in the kidneys. Any TTR or RBP molecules that are filtered are rapidly bound to megalin, the multiligand receptor expressed on the luminal surface of the renal proximal tubules and therefore internalized. Thus, under physiological conditions, TTR and RBP are present in urine if at all, only in trace amounts. The gene TTR that encodes transthyretin is in chromosome region 18q11.2-q12.1.

Transthyretin has been associated with Alzheimer's disease and depression¹⁷. It has also been shown that schizophrenia patients treated with clozapine show differences in transthyretin levels¹⁸.

Apolipoproteins function in lipid transport as structural components of lipoprotein particles, co-factors for enzymes and ligands for cell-surface receptors. There are five major types of apolipoproteins: ApoA (ApoA1, ApoA 2), ApoB, ApoC (ApoC1, ApoC2, ApoC3, ApoC4), ApoD, and ApoE. In particular, ApoA1 is the major protein component of high-density lipoproteins; ApoA4 is thought to act primarily in intestinal lipid absorption; and ApoE is a blood plasma protein that mediates the transport and uptake of cholesterol and lipid by way of its high affinity interaction with different cellular receptors, including the low-density lipoprotein (LDL) receptor. ApoA1 is known to have cardio-protective properties and play a role in atherosclerosis and diabetes^{28,29}.

Wen et al³⁰ discloses that the level of ApoA1 in patients which have undergone therapy with phenothiazine is lower compared to normal controls from healthy individuals.

Middleton et al³¹ analysed the expression levels of the Apolipoprotein gene family cluster.

### SUMMARY OF THE INVENTION

The present invention is based in part on the results of ¹H NMR-based metabonomics approach to profile plasma from identical twins discordant for the psychotic disorder schizophrenia (i.e., with one affected twin and one non-affected twin) and from healthy control sets of twins, to identify a disease-related metabolic signature.

In one aspect, the invention provides a method of diagnosing or monitoring a psychotic disorder in a subject, comprising:

(a) providing a test biological sample from said subject,
(b) performing spectral analysis on said test biological sample to provide one or more spectra, and
(c) comparing said one or more spectra with one or more control spectra.

The invention further provides a method of diagnosing or monitoring a psychotic disorder in a subject, comprising:

(a) providing a test biological sample from said subject,
(b) performing spectral analysis on said test biological sample to provide one or more spectra,
(c) analysing said one or more spectra to detect the level of one or more biomarkers in said spectra, and
(d) comparing the level of said one or more biomarkers in said one or more spectra with the level of said one or more biomarkers detected in control spectra.

In a further aspect, the invention provides a method of diagnosing or monitoring a psychotic disorder, or predisposition thereto, comprising measuring the level of one or more biomarkers present in a biological sample taken from a test subject, said biomarkers being selected from: VLDL, LDL and aromatic species such as plasma proteins. Such methods can be used in methods of monitoring efficacy of a therapy (e.g. a therapeutic substance) in a subject having, suspected of having, or of being predisposed to, a psychotic disorder.

In a further aspect, the invention provides a multi-analyte panel or array capable of detecting one, two or three biomarkers selected from the group: VLDL, LDL and aromatic species such as plasma proteins.

A multi-analyte panel is capable of detecting a number of different analytes. An array can be capable of detecting a single analyte in a number of samples or, as a multi-analyte array, can be capable of detecting a number of different analytes in a sample. A multi-analyte panel or multi-analyte array according to the invention is capable of detecting one or more metabolic biomarker as described herein, and can be capable of detecting a biomarker or biomarkers additional to those specifically described herein.

Also provided is a diagnostic or monitoring test kit suitable for performing a method according to the invention, optionally together with instructions for use of the kit. The diagnostic or monitoring kit may comprise one or more biosensors according to the invention, a single sensor, or biosensor or combination of sensors and/or biosensors may be included in the kit. A diagnostic or monitoring kit may comprise a panel or an array according to the invention. A diagnostic or monitoring kit may comprise an assay or combination of assays according to the invention.

Further provided is the use of one or more biomarkers selected from VLDL, LDL and aromatic species such as plasma proteins, to diagnose and/or monitor a psychotic disorder.

Yet further provided is the use of a method, sensor, biosensor, multi-analyte panel, array or kit according to the invention to identify a substance capable of modulating a psychotic disorder. A substance capable of modulating a psychotic disorder may be an anti-psychotic substance useful for treatment of psychoses, or a pro-psychotic substance which may induce psychoses.

Additionally provided is a method of identifying a substance capable of modulating a psychotic disorder in a subject, comprising a method of monitoring as described herein; particularly preferred identification methods comprise administering a test substance to a test subject and detecting the level of one or more biomarkers selected from VLDL, LDL and aromatic species such as plasma proteins in a biological sample, preferably a whole blood, serum or plasma sample taken from said subject.

The invention also relates to the use of a transthyretin peptide comprising the amino acid sequence shown in SEQ ID NO: 1 or a fragment thereof as a biomarker for a schizophrenic disorder or predisposition thereto.

The invention further provides a transthyretin peptide biomarker for a schizophrenic disorder comprising the amino acid sequence shown in SEQ ID NO: 1 or a fragment thereof.

In a further aspect, the invention provides a method of diagnosing or monitoring a schizophrenic disorder or predisposition thereto, comprising detecting and/or quantifying a transthyretin peptide biomarker comprising the amino acid sequence of SEQ ID NO: 1 or a fragment thereof, present in a biological sample from a test subject.

A further aspect of the invention provides ligands, such as naturally occurring or chemically synthesised compounds, capable of specific binding to the transthyretin peptide biomarker. A ligand according to the invention may comprise a peptide, an antibody or a fragment thereof, or an aptamer or oligonucleotide, capable of specific binding to the transthyretin peptide biomarker. The antibody can be a monoclonal antibody or a fragment thereof capable of specific binding to the transthyretin peptide biomarker. A ligand according to the invention may be labelled with a detectable marker, such as a luminescent, fluorescent, enzyme or radioactive marker; alternatively or additionally a ligand according to the invention may be labelled with an affinity tag. Preferably, a ligand according to the invention comprises a peptide, an antibody or a fragment thereof, or an aptamer or oligonucleotide, capable of specific binding to a transthyretin peptide biomarker as described herein wherein the ligand is not an antibody selected from the group as listed in Table 1 or a ligand selected from the group comprising T3, T4 (thyroid hormones), vitamin A (indirectly by interacting with serum retinol-binding protein), apolipoprotein AI (ApoAI), noradrenaline oxidation products, and pterins, non-steroidal anti-inflammatory drugs (NSAIDs), environmental pollutants, such as polyhalogenated biphenyls and thyromimetic compounds, xanthone derivatives or natural and synthetic flavonoids.

The present invention provides a method of diagnosing a schizophrenic disorder or predisposition thereto, comprising detecting and/or quantifying in a biological sample from a test subject an ApoA1 peptide biomarker comprising the amino acid sequence of SEQ ID NO: 2 or a fragment thereof.

Biomarkers for schizophrenic disorders are targets for discovery of novel targets and drug molecules that retard or halt disease progression. As the level of an ApoA1 peptide biomarker is indicative of disorder and of drug response, the ApoA1 biomarker is useful for identification of novel therapeutic compounds in in vitro and/or in vivo assays. The ApoA1 biomarker of the invention can therefore be employed in methods for screening for compounds that promote the activity of, or activate the generation of, an ApoA1 peptide biomarker according to the invention.

Thus, in a further aspect of the invention, there is provided the use of a substance or ligand capable of stimulating or promoting the generation of an ApoA1 biomarker peptide said biomarker comprising the amino acid sequence of SEQ ID NO: 2 or a fragment thereof in the manufacture of a medicament for the treatment of a schizophrenic disorder or predisposition thereto. Also provided is the use of a substance or ligand capable of stimulating the activity of an ApoA1 biomarker peptide, said biomarker comprising the amino acid sequence of SEQ ID NO: 2 or a fragment thereof in the manufacture of a medicament for the treatment of a schizophrenic disorder or predisposition thereto.

The invention also relates to a method for treating a schizophrenic disorder comprising administering to a patient in need thereof a substance or ligand capable of stimulating, promoting or activating the activity or the generation of a peptide comprising the amino acid sequence of SEQ ID NO: 2 or a fragment thereof.

A lower level of plasma protein biomarkers in the test biological sample relative to the level in a normal control is indicative of the presence of a psychotic disorder, in particular a schizophrenic disorder, bipolar disorder, or predisposition thereto.

Methods of monitoring and of diagnosis according to the invention are useful to confirm the existence of a disorder, or predisposition thereto; to monitor development of the disorder by assessing onset and progression, or to assess amelioration or regression of the disorder. Methods of monitoring and of diagnosis are also useful in methods for assessment of clinical screening, prognosis, choice of therapy, evaluation of therapeutic benefit, i.e. for drug screening and drug development.

Efficient diagnosis and monitoring methods provide very powerful “patient solutions” with the potential for improved prognosis, by establishing the correct diagnosis, allowing rapid identification of the most appropriate treatment (thus lessening unnecessary exposure to harmful drug side effects), reducing “down-time” and relapse rates.

Methods for monitoring efficacy of a therapy can be used to monitor the therapeutic effectiveness of existing therapies and new therapies in human subjects and in non-human animals (e.g. in animal models). These monitoring methods can be incorporated into screens for new drug substances and combinations of substances

Modulation of a peptide biomarker level is useful as an indicator of the state of the schizophrenic disorder or predisposition thereto. A decrease in the level of peptide biomarker over time is indicative of onset or progression, i.e. worsening of the disorder, whereas an increase in the level of peptide biomarker indicates amelioration or remission of the disorder.

The identification of biomarkers for schizophrenic disorders permits integration of diagnostic procedures and therapeutic regimes. Currently there are significant delays in determining effective treatment and it has not hitherto been possible to perform rapid assessment of drug response. Traditionally, many anti-schizophrenic therapies have required treatment trials lasting weeks to months for a given therapeutic approach. Detection of a peptide biomarker of the invention can be used to screen subjects prior to their participation in clinical trials. The biomarker provides a means to indicate therapeutic response, failure to respond, unfavourable side-effect profile, degree of medication compliance and achievement of adequate serum drug levels. The biomarker may be used to provide warning of adverse drug response, a major problem encountered with all psychotropic medications. Biomarkers are useful in development of personalized brain therapies, as assessment of response can be used to fine-tune dosage, minimise the number of prescribed medications, reduce the delay in attaining effective therapy and avoid adverse drug reactions. Thus by monitoring a biomarker of the invention, patient care can be tailored precisely to match the needs determined by the disorder and the pharmacogenomic profile of the patient, the biomarker can thus be used to titrate the optimal dose, predict a positive therapeutic response and identify those patients at high risk of severe side effects.

Biomarker based tests provide a first line assessment of ‘new’ patients, and provide objective measures for accurate and rapid diagnosis, in a time frame and with precision, not achievable using the current subjective measures.

Furthermore, diagnostic biomarker tests are useful to identify family members or patients in the “prodromal phase”, i.e. those at high risk of developing overt schizophrenia. This permits initiation of appropriate therapy, for example low dose antipsychotics, or preventive measures, e.g. managing risk factors such as stress, illicit drug use or viral infections. These approaches are recognised to improve outcome and may prevent overt onset of the disorder.

Biomarker monitoring methods, biosensors and kits are also vital as patient monitoring tools, to enable the physician to determine whether relapse is due to a genuine breakthrough or worsening of the disease, poor patient compliance or substance abuse. If pharmacological treatment is assessed to be inadequate, then therapy can be reinstated or increased. For genuine breakthrough disease, a change in therapy can be given if appropriate. As the biomarker is sensitive to the state of the disorder, it provides an indication of the impact of drug therapy or of substance abuse.

High-throughput screening technologies based on the biomarkers of the invention, uses and methods of the invention, e.g. configured in an array format, are suitable to monitor biomarkers for the identification of potentially useful therapeutic compounds, e.g. ligands such as natural compounds, synthetic chemical compounds (e.g. from combinatorial libraries), peptides, monoclonal or polyclonal antibodies or fragments thereof, capable of modulating the expression of the biomarkers.

### Sequence Listing Information

SEQ ID NO:1

Amino acid sequence of human transthyretin
PLMVKVLDAV RGSPAINVAV HVFDKAADDT WEPFASGKTS
ESGELHGLTT EEEFVEGIYK VEIDTKSYWK ALGISPFHEH
AEVVFTANDS GPRRYTIAAL LSPYSYSTTA VVTNPKE

SEQ ID No:2

ApoA1

| | |
|---|---|
| 1 | mkaavltlav lfltgsqarh fwqqdeppqs pwarvkdlat |
| | vyvdvlkdsg rdyvsqfegs |
| 61 | algkqlnlkl ldnwdsvtst fsklreqlgp vtqefwdnle |
| | keteglrqem skdleevkak |
| 121 | vqpylddfqk kwqeemelyr qkveplrael qegarqklhe |
| | lqeklsplge emrdrarahv |
| 181 | dalrthlapy sdelrqrlaa rlealkengg arlaeyhaka |
| | tehlstlsek akpaledlrq |
| 241 | gllpvlesfk vsflsaleey tkklntq |

Two or more biomarkers described herein may be used in combination. Each aspect of the invention, as described herein, with respect to a particular biomarker, may be equally applicable to any other biomarker described herein. Further, any reference to schizophrenia may equally apply to another psychotic.

### DESCRIPTION OF THE INVENTION

The term “biomarker” means a distinctive biological or biologically derived indicator of a process, event, or condition. Peptide biomarkers can be used in methods of diagnosis, e.g. clinical screening, and prognosis assessment; and in monitoring the results of therapy, for identifying patients most likely to respond to a particular therapeutic treatment, as well as in drug screening and development. Biomarkers and uses thereof are valuable for identification of new drug treatments and for discovery of new targets for drug treatment.

The term transthyretin peptide biomarker includes the mature full length human transthyretin peptide. A preferred transthyretin peptide biomarker (SEQ ID NO: 1) is, or is derived from, the human transthyretin protein. The peptide of SEQ ID NO: 1 is the secreted form which does not include the signal (leader) sequence as found in the precursor. Also included are transthyretin isoforms and derivatives, for example S-cysteinylated and S-gluthanionylated transthyretin, which are both common modifications of TTR found in CSF samples. The peptide biomarker as shown in SEQ ID NO: 1 is found to be present at lower levels in individuals with first onset psychosis characteristic of schizophrenia, it is thus useful as a marker for diagnosing and monitoring schizophrenic disorders or predisposition thereto. According to the invention, the biomarker may comprise the amino acid sequence shown in SEQ ID NO: 1 or a fragment thereof. For example, the biomarker may comprise one or more fragments (multiple fragments) of the amino acid sequence shown in SEQ ID NO: 1.

The term ApoA1 peptide biomarker includes the mature full length human ApoA1 peptide. A preferred ApoA1 peptide biomarker is shown in SEQ ID NO: 1. The peptide biomarker as shown in SEQ ID NO: 2 (FIG. 13) is found to be present at decreased levels in drug-naïve individuals with first-onset psychosis characteristic of schizophrenic disorders compared to normal controls. It is thus useful as a marker for diagnosing schizophrenic disorders, or predisposition thereto.

The term drug-naïve patient as used herein means an individual who has not been treated with any schizophrenia therapeutic substance. Thus in a preferred embodiment, the invention relates to a method wherein the test sample is from a test subject wherein the test subject is a first onset drug-naïve individual, and the sample is taken prior to administration of any anti-schizophrenic therapy to the subject. The control sample is preferably a sample from a normal individual.

A lower level of the ApoA1 peptide biomarker in a test sample relative to the level in a normal control is indicative of the presence of a schizophrenic disorder or predisposition thereto. An equivalent or higher level of said peptide in the test sample relative to the normal control is indicative of the absence of a schizophrenic disorder or a predisposition thereto.

The term “diagnosis” as used herein encompasses identification, confirmation, and/or characterisation of a schizophrenic disorder or predisposition thereto. The term “predisposition” as used herein means that a subject does not currently present with the disorder, but is liable to be affected by the disorder in time. Methods of diagnosis according to the invention are useful to confirm the existence of a disorder, or predisposition thereto. Methods of diagnosis are also useful in methods for assessment of clinical screening, prognosis, choice of therapy, evaluation of therapeutic benefit, i.e. for drug screening and drug development.

Monitoring methods of the invention can be used to monitor onset, progression, stabilisation, amelioration and/or remission of a psychotic disorder.

The term “psychotic disorder” as used herein refers to a disorder in which psychosis is a recognised symptom, this includes neuropsychiatric (psychotic depression and other psychotic episodes) and neurodevelopmental disorders (especially Autistic spectrum disorders), neurodegenerative disorders, depression, mania, and in particular, schizophrenic disorders (paranoid, catatonic, disorganized, undifferentiated and residual schizophrenia) and bipolar disorders.

Biological samples that may be tested in a method of the invention include whole blood, blood serum or plasma, urine, saliva, cerebrospinal fluid (CSF) or other bodily fluid (stool, tear fluid, synovial fluid, sputum), breath, e.g. as condensed breath, or an extract or purification therefrom, or dilution thereof. Biological samples also include tissue homogenates, tissue sections and biopsy specimens from a live subject, or taken post-mortem. The samples can be prepared, for example where appropriate diluted or concentrated, and stored in the usual manner.

A number of spectroscopic techniques can be used to generate spectra, according to the invention, including NMR spectroscopy and mass spectrometry. In preferred methods, spectral analysis is performed by NMR spectroscopy, preferably ¹H NMR spectroscopy. One or more spectra may be generated, a suite of spectra may be measured, including one for small molecules and another for macromolecule profiles. The spectra obtained may be subjected to spectral editing techniques. One or two-dimensional NMR spectroscopy may be performed.

An advantage of using NMR spectroscopy to study complex biomixtures is that measurements can often be made with minimal sample preparation (usually with only the addition of 5-10% D₂O) and a detailed analytical profile of the whole biological sample can be obtained.

Sample volumes are small, typically 0.3 to 0.5 ml for standard probes, and as low as 3 μl for microprobes. Acquisition of simple NMR spectra is rapid and efficient using flow-injection technology. It is usually necessary to suppress the water NMR resonance.

High resolution NMR spectroscopy (in particular ¹H NMR) is particularly appropriate. The main advantages of using ¹H NMR spectroscopy are the speed of the method (with spectra being obtained in 5 to 10 minutes), the requirement for minimal sample preparation, and the fact that it provides a non-selective detector for all metabolites in the biofluid regardless of their structural type, provided only that they are present above the detection limit of the NMR experiment and that they contain non-exchangeable hydrogen atoms.

NMR studies of body fluids should ideally be performed at the highest magnetic field available to obtain maximal dispersion and sensitivity and most ¹H NMR studies are performed at 400 MHz or greater, e.g. 600 MHz.

Usually, to assign ¹H NMR spectra, comparison is made with control spectra of authentic materials and/or by standard addition of an authentic reference standard to the sample. The control spectra employed may be normal control spectra, generated by spectral analysis of a biological sample from a normal subject, and/or psychotic disorder control spectra, generated by spectral analysis of a biological sample from a subject with a psychotic disorder.

Additional confirmation of assignments is usually sought from the application of other NMR methods, including, for example, 2-dimensional (2D) NMR methods, particularly COSY (correlation spectroscopy), TOCSY (total correlation spectroscopy), inverse-detected heteronuclear correlation methods such as HMBC (heteronuclear multiple bond correlation), HSQC (heteronuclear single quantum coherence), and HMQC (heteronuclear multiple quantum coherence), 2D J-resolved (JRES) methods, spin-echo methods, relaxation editing, diffusion editing (e.g., both 1D NMR and 2D NMR such as diffusion-edited TOCSY), and multiple quantum filtering.

By comparison of spectra with normal and/or psychotic disorder control spectra, the test spectra can be classified as having a normal profile, a psychotic disorder profile, or a psychotic disorder predisposition profile.

Comparison of spectra may be performed on entire spectra or on selected regions of spectra. Comparison of spectra may involve an assessment of the variation in spectral regions responsible for deviation from the normal spectral profile and in particular, assessment of variation in one or more biomarkers within those regions.

A limiting factor in understanding the biochemical information from both 1D and 2D-NMR spectra of biofluids, such as plasma, is their complexity. The most efficient way to compare and investigate these complex multiparametric data is employ the 1D or 2D NMR metabonomic approach in combination with computer-based “pattern recognition” (PR) methods and expert systems.

Although the utility of the metabonomic approach is well established, its full potential has not yet been exploited. The metabolic variation is often subtle, and powerful analysis methods are required for detection of particular analytes, especially when the data (e.g., NMR spectra) are so complex.

Metabonomics methods (which employ multivariate statistical analysis and pattern recognition (PR) techniques, and optionally data filtering techniques) of analysing data (e.g. NMR spectra) from a test population yield accurate mathematical models which may subsequently be used to classify a test sample or subject, and/or in diagnosis.

Comparison of spectra may include one or more chemometric analyses of the spectra. The term “chemometrics” is applied to describe the use of pattern recognition (PR) methods and related multivariate statistical approaches to chemical numerical data. Comparison may therefore comprise one or more pattern recognition analysis methods, which can be performed by one or more supervised and/or unsupervised methods.

Pattern recognition (PR) methods can be used to reduce the complexity of data sets, to generate scientific hypotheses and to test hypotheses. In general, the use of pattern recognition algorithms allows the identification, and, with some methods, the interpretation of some non-random behaviour in a complex system which can be obscured by noise or random variations in the parameters defining the system. Also, the number of parameters used can be very large such that visualisation of the regularities or irregularities, which for the human brain is best in no more than three dimensions, can be difficult.

Usually the number of measured descriptors is much greater than three and so simple scatter plots cannot be used to visualise any similarity or disparity between samples. Pattern recognition methods have been used widely to characterise many different types of problem ranging for example over linguistics, fingerprinting, chemistry and psychology.

In the context of the methods described herein, pattern recognition is the use of multivariate statistics, both parametric and non-parametric, to analyse spectroscopic data, and hence to classify samples and to predict the value of some dependent variable based on a range of observed measurements. There are two main approaches. One set of methods is termed “unsupervised” and these simply reduce data complexity in a rational way and also produce display plots which can be interpreted by the human eye. The other approach is termed “supervised” whereby a training set of samples with known class or outcome is used to produce a mathematical model and this is then evaluated with independent validation data sets.

Unsupervised techniques are used to establish whether any intrinsic clustering exists within a data set and consist of methods that map samples, often by dimension reduction, according to their properties, without reference to any other independent knowledge, e.g. without prior knowledge of sample class. Examples of unsupervised methods include principal component analysis (PCA), non-linear mapping (NLM) and clustering methods such as hierarchical cluster analysis.

One of the most useful and easily applied unsupervised PR techniques is principal components analysis (PCA) (see, for example, Kowalski et al., 1986). Principal components (PCs) are new variables created from linear combinations of the starting variables with appropriate weighting coefficients. The properties of these PCs are such that: (i) each PC is orthogonal to (uncorrelated with) all other PCs, and (ii) the first PC contains the largest part of the variance of the data set (information content) with subsequent PCs containing correspondingly smaller amounts of variance.

PCA, a dimension reduction technique, takes m objects or samples, each described by values in K dimensions (descriptor vectors), and extracts a set of eigenvectors, which are linear combinations of the descriptor vectors. The eigenvectors and eigenvalues are obtained by diagonalisation of the covariance matrix of the data. The eigenvectors can be thought of as a new set of orthogonal plotting axes, called principal components (PCs). The extraction of the systematic variations in the data is accomplished by projection and modelling of variance and covariance structure of the data matrix. The primary axis is a single eigenvector describing the largest variation in the data, and is termed principal component one (PC1). Subsequent PCs, ranked by decreasing eigenvalue, describe successively less variability. The variation in the data that has not been described by the PCs is called residual variance and signifies how well the model fits the data. The projections of the descriptor vectors onto the PCs are defined as scores, which reveal the relationships between the samples or objects. In a graphical representation (a “scores plot” or eigenvector projection), objects or samples having similar descriptor vectors will group together in clusters. Another graphical representation is called a loadings plot, and this connects the PCs to the individual descriptor vectors, and displays both the importance of each descriptor vector to the interpretation of a PC and the relationship among descriptor vectors in that PC. In fact, a loading value is simply the cosine of the angle which the original descriptor vector makes with the PC.

Descriptor vectors which fall close to the origin in this plot carry little information in the PC, while descriptor vectors distant from the origin (high loading) are important for interpretation.

Thus a plot of the first two or three PC scores gives the “best” representation, in terms of information content, of the data set in two or three dimensions, respectively. A plot of the first two principal component scores, PC1 and PC2 provides the maximum information content of the data in two dimensions. Such PC maps can be used to visualise inherent clustering behaviour, for example, for drugs and toxins based on similarity of their metabonomic responses and hence mechanism of action. Of course, the clustering information may be in lower PCs and these can also be examined.

Hierarchical Cluster Analysis, another unsupervised pattern recognition method, permits the grouping of data points which are similar by virtue of being “near” to one another in some multidimensional space. Individual data points may be, for example, the signal intensities for particular assigned peaks in an NMR spectrum. A “similarity matrix” S, is constructed with element ssij=1−rij/rijmax′ where rij is the interpoint distance between points i and j (e.g., Euclidean interpoint distance), and rijmax is the largest interpoint distance for all points.

The most distant pair of points will have sij equal to 0, since rij then equals rijmaX. Conversely, the closest pair of points will have the largest sij, approaching 1. The similarity matrix is scanned for the closest pair of points. The pair of points is reported with their separation distance, and then the two points are deleted and replaced with a single combined point. The process is then repeated iteratively until only one point remains. A number of different methods may be used to determine how two clusters will be joined, including the nearest neighbour method (also known as the single link method), the furthest neighbour method, the centroid method (including centroid link, incremental link, median link, group average link, and flexible link variations).

The reported connectivities are then plotted as a dendrogram (a tree-like chart which allows visualisation of clustering), showing sample-sample connectivities versus increasing separation distance (or equivalently, versus decreasing similarity). In the dendrogram the branch lengths are proportional to the distances between the various clusters and hence the length of the branches linking one sample to the next is a measure of their similarity. In this way, similar data points may be identified algorithmically.

Supervised methods of analysis use the class information given for a training set of sample data to optimise the separation between two or more sample classes. These techniques include soft independent modelling of class analogy, partial least squares (PLS) methods, such as projection to latent discriminant analysis (PLS DA), k-nearest neighbour analysis and neural networks. Neural networks are a non-linear method of modelling data. A training set of data is used to develop algorithms that ‘learn’ the structure of the data and can cope with complex functions. Several types of neural network have been applied successfully to predicting toxicity or disease from spectral information.

Statistical techniques such as one-way analysis of variance (ANOVA) may also be employed to analyse data.

Methods of the invention involving spectral analysis this may be performed to provide spectra from biological samples taken on two or more occasions from a test subject. Spectra from biological samples taken on two or more occasions from a test subject can be compared to identify differences between the spectra of samples taken on different occasions. Methods may include analysis of spectra from biological samples taken on two or more occasions from a test subject to quantify the level of one or more biomarkers present in the biological samples, and comparing the level of the one or more biomarkers present in biological samples taken on two or more occasions.

Diagnostic and monitoring methods of the invention are useful in methods of assessing prognosis of a psychotic disorder, in methods of monitoring efficacy of an administered therapeutic substance in a subject having, suspected of having, or of being predisposed to, a psychotic disorder and in methods of identifying an anti-psychotic or pro-psychotic substance. Such methods may comprise comparing the level of the one or more biomarkers in a test biological sample taken from a test subject with the level present in one or more samples taken from the test subject prior to administration of the substance, and/or one or more samples taken from the test subject at an earlier stage during treatment with the substance. Additionally, these methods may comprise detecting a change in the level of the one or more biomarkers in biological samples taken from a test subject on two or more occasions.

In methods of the invention, in particular those in which spectral analysis is employed, and in particular when the biological sample is blood or is derived from blood, e.g. plasma or serum, suitably one or more biomarkers is selected from: VLDL, LDL and aromatic species such as plasma proteins.

In a ¹H NMR-based metabonomics study, alterations in the lipid profile of both affected and unaffected schizophrenia twins have been identified. Lipid levels were found to correlate strongly with global function scores for affected female twins.

These biomarkers of psychotic disorder, in particular schizophrenic disorders, were identified by extensive metabolic profiling analysis using ¹H NMR spectroscopy in conjunction with computerised pattern recognition analysis to investigate plasma samples from 21 pairs of monozygotic twins discordant for schizophrenia and 8 pairs of age-matched healthy control twins. All samples were obtained under standardized conditions and were annotated with regards to demographic and clinical details.

The results of these studies show that signals from VLDL, LDL and aromatic regions relating to plasma proteins, are the most important factors differentiating ill and healthy co-twins discordant for schizophrenia from healthy control twins. VLDL and LDL levels were found to be elevated in twins discordant for schizophrenia compared to normal control twins without schizophrenia. In the discordant twins, the affected twins had VLDL and LDL levels that were more elevated than the levels found in the unaffected discordant twins. This differentiation was very pronounced in female twins. A close association of VLDL/LDL signals and Global Functioning Scores (DSMIV, Axis V) was found in female twins suffering from schizophrenia. Discordant twins had lower plasma protein levels than normal control co-twins, the greatest reductions in plasma protein being found in the affected twins.

The observed changes in the lipid and aromatic region in twins discordant for schizophrenia suggests a link between metabolic disturbances and the aetiopathology of schizophrenia. Although effects of antipsychotic medication can not be ruled out entirely, the fact that healthy co-twins show a putative “predisposition” signature implies that these changes are disease-related, rather than an artifact of medication. The increase in VLDL, LDL and decrease in aromatic regions (plasma proteins) constitute metabolic biomarkers that enable differentiation between normal individuals and those with a psychotic disorder.

Lipid profiles of affected female twins were also found to correlate highly with Global Functioning Scores (GAF). GAF scores are based on subjective assessment by a psychiatrist. The correlation between elevation of VLDL and LDL lipid biomarker levels and GAF score provides an objective means to confirm and validate the subjective GAF score for diagnosis and monitoring of psychotic disease such as schizophrenia (and bipolar disorder).

Methods of diagnosing or monitoring according to the invention, may comprise measuring the level of one or more of the biomarkers present in biological samples taken on two or more occasions from a test subject. Comparisons may be made between the level of the biomarkers in samples taken on two or more occasions. Assessment of any change in the level of the biomarkers in samples taken on two or more occasions may be performed. Modulation of the biomarker level is useful as an indicator of the state of the psychotic disorder or predisposition thereto.

An increase in the level of VLDL or LDL in a biological sample, preferably in plasma, over time is indicative of onset or progression, i.e. worsening of the disorder, whereas a decrease in the level of VLDL or LDL indicates amelioration or remission of the disorder.

A decrease in the level of plasma protein in a biological sample, preferably in a sample of whole blood, plasma, or serum over time is indicative of onset or progression, i.e. worsening of the disorder, whereas an increase in the level of plasma protein indicates amelioration or remission of the disorder.

A method according to the invention may comprise comparing the level of one or more biomarkers in a biological sample taken from a test subject with the level present in one or more samples taken from the test subject prior to commencement of a therapy, and/or one or more samples taken from the test subject at an earlier stage of a therapy. Such methods may comprise detecting a change in the amount of the one or more biomarkers in samples taken on two or more occasions. Methods of the invention are particularly useful in assessment of anti-psychotic therapies.

A method of diagnosis of or monitoring according to the invention may comprise quantifying the one or more biomarkers in a test biological sample taken from a test subject and comparing the level of the one or more biomarkers present in said test sample with one or more controls. The control can be selected from a normal control and/or a psychotic disorder control. The control used in a method of the invention can be one or more controls selected from the group consisting of: the level of biomarker found in a normal control sample from a normal subject, a normal biomarker level; a normal biomarker range, the level in a sample from a subject with a schizophrenic disorder, bipolar disorder, related psychotic disorder, or a diagnosed predisposition thereto; a schizophrenic disorder marker level, a bipolar disorder marker level, a related psychotic disorder marker level, a schizophrenic disorder marker range, a bipolar disorder marker range and a related psychotic disorder marker range.

Biological samples can be taken at intervals over the remaining life, or a part thereof, of a subject. Suitably, the time elapsed between taking samples from a subject undergoing diagnosis or monitoring will be 3 days, 5 days, a week, two weeks, a month, 2 months, 3 months, 6 or 12 months. Samples may be taken prior to and/or during and/or following an anti-psychotic therapy, such as an anti-schizophrenic or anti-bipolar disorder therapy.

Measurement of the level of a biomarker can be performed by any method suitable to identify the amount of the biomarker in a biological sample taken from a patient or a purification of or extract from the sample or a dilution thereof. Measuring the level of a biomarker present in a sample may include determining the concentration of the biomarker present in the sample. Such quantification may be performed directly on the sample, or indirectly on an extract therefrom, or on a dilution thereof. In methods of the invention, in addition to measuring the concentration of the biomarker in a biological sample, which is preferably whole blood, plasma or serum, the concentration of the biomarker may be tested in a different biological sample taken from the test subject, e.g. CSF, urine, saliva, or other bodily fluid (stool, tear fluid, synovial fluid, sputum), breath, e.g. as condensed breath, or an extract or purification therefrom, or dilution thereof. Biological samples also include tissue homogenates, tissue sections and biopsy specimens from a live subject, or taken post-mortem. The samples can be prepared, for example where appropriate diluted or concentrated, and stored in the usual manner.

Biomarker levels can be measured by one or more methods selected from the group consisting of: spectroscopy methods such as NMR (nuclear magnetic resonance), or mass spectroscopy (MS); SELDI (-TOF), MALDI (-TOF), a 1-D gel-based analysis, a 2-D gel-based analysis, liquid chromatography (e.g. high pressure liquid chromatography (HPLC) or low pressure liquid chromatography (LPLC)), thin-layer chromatography, and LC-MS-based techniques. Appropriate LC MS techniques include ICAT® (Applied Biosystems, CA, USA), or iTRAQ® (Applied Biosystems, CA, USA).

Measurement of a biomarker may be performed by a direct or indirect detection method. A biomarker may be detected directly, or indirectly, via interaction with a ligand or ligands, such as an enzyme, binding receptor or transporter protein, antibody, peptide, aptamer, or oligonucleotide, or any synthetic chemical receptor or compound capable of specifically binding the biomarker. The ligand may possess a detectable label, such as a luminescent, fluorescent or radioactive label and/or an affinity tag.

The term “antibody” as used herein includes, but is not limited to: polyclonal, monoclonal, bispecific, humanised or chimeric antibodies, single chain antibodies, Fab fragments and F (ab′)₂ fragments, fragments produced by a Fab expression library, anti-idiotypic (anti-Id) antibodies, and epitope-binding fragments of any of the above. The term “antibody” as used herein also refers to immunoglobulin molecules and immunologically-active portions of immunoglobulin molecules, i.e., molecules that contain an antigen binding site that specifically binds an antigen. The immunoglobulin molecules of the invention can be of any class (e.g., IgG, IgE, IgM, IgD and IgA) or subclass of immunoglobulin molecule.

Metabolite biomarkers as described herein are suitably measured by conventional chemical or enzymatic methods (which may be direct or indirect and or may not be coupled), electrochemical, fluorimetric, luminometric, spectrophotometric, fluorimetric, luminometric, spectrometric, polarimetric, chromatographic (e.g. HPLC) or similar techniques.

For enzymatic methods, consumption of a substrate in the reaction, or generation of a product of the reaction, may be detected, directly or indirectly, as a means of measurement.

VLDL and LDL biomarkers can be detected and levels measured using various detection systems including liquid-phase chemical methods (immunoseparation and separation with polyanion surfactant/detergent combinations), physical methods for separation of lipoproteins (e.g., electrophoresis, capillary isotachophoresis, and chromatography), which may be performed in conjunction with enzymatic assays e.g. the cholesterol esterase-cholesterol oxidase (peroxidase) enzymatic assay, as well as indirect methods such as NMR.

In normal individuals VLDL and LDL levels in serum/plasma are generally 85 mg/dl+/−15% and 30 mg/dl+/−10% for VLDL and LDL respectively, thus levels above these are diagnostic of psychotic disorder, especially schizophrenia, a bipolar disorder, or a predisposition thereto.

Aromatic species biomarkers such as plasma proteins can be detected and levels measured using methods including, but not limited to, ultraviolet absorbance and calorimetric methods such as Bradford assay, Lowry assay, and BCA assay.

The biomarkers of the invention are preferably detected and measured using mass spectrometry-based techniques; chromatography-based techniques; enzymatic detection systems (by direct or indirect measurements); or using sensors, e.g. with sensor systems with amperometric, potentiometric, conductimetric, impedance, magnetic, optical, acoustic or thermal transducers.

A sensor may incorporate a physical, chemical or biological detection system. An example of a sensor is a biosensor, i.e. a sensor with a biological recognition system, e.g. based on a nucleic acid, such as an oligonucleotide probe or aptamer, or a protein such as an enzyme, binding protein, receptor protein, transporter protein or antibody.

The biosensor may incorporate an immunological method for detection of the biomarker, an electrical, thermal, magnetic, optical (e.g. hologram) or acoustic technologies. Using such biosensors, it is possible to detect the target biomarker at the anticipated concentrations found in biological samples.

Methods of the invention are suitable for clinical screening, assessment of prognosis, monitoring the results of therapy, identifying patients most likely to respond to a particular therapeutic treatment, for drug screening and development, and to assist in identification of new targets for drug treatment. The identification of key biomarkers specific to a disease is central to integration of diagnostic procedures and therapeutic regimes.

Methods of the invention may further comprise one or more assessments to diagnose and/or monitor a psychotic disorder in a subject. Assessment may be a clinical assessment, carried out by a clinician in accordance with accepted assessment protocols, e.g. global functioning score (GAF) or SCID, and/or may involve a self-assessment by the subject. Rating scales may be used to assist diagnosis and/or monitoring. GAF and SCID are assessed on the basis of a clinical interview. It is preferred that assessments, such as global functioning score, are made at (i.e. the same day as) or around (i.e. within a few days of) the time of collection of the test biological sample from the subject. This is particularly useful as a tool for diagnosing and monitoring female subjects, in which VLDL and LDL levels were found to have a very close inverse correlation with the clinical assessment as determined by global functioning score.

Using predictive biomarkers such as those described herein, appropriate diagnostic tools such as sensors and biosensors can be developed, accordingly, in methods and uses of the invention, detecting and quantifying one or more biomarkers can be performed using a sensor or biosensor.

A sensor or biosensor according to the invention is a psychotic disorder sensor or biosensor capable of quantifying one, two, or three biomarkers selected from the group: VLDL, LDL and aromatic species such as plasma proteins.

The sensor or biosensor may incorporate detection methods and systems as described herein for detection of the biomarker. Sensors or biosensors may employ electrical (e.g. amperometric, potentiometric, conductimetric, or impedance detection systems), thermal (e.g. transducers), magnetic, optical (e.g. hologram) or acoustic technologies. In a sensor or biosensor according to the invention the level of one, two, or three biomarkers can be detected by one or more methods selected from: direct, indirect or coupled enzymatic, spectrophotometric, fluorimetric, luminometric, spectrometric, polarimetric and chromatographic techniques. Particularly preferred sensors or biosensors comprise one or more enzymes used directly or indirectly via a mediator, or using a binding, receptor or transporter protein, coupled to an electrical, optical, acoustic, magnetic or thermal transducer. Using such biosensors, it is possible to detect the level of target biomarkers at the anticipated concentrations found in biological samples.

A biomarker or biomarkers of the invention can be detected using a sensor or biosensor incorporating technologies based on “smart” holograms, or high frequency acoustic systems, such systems are particularly amenable to “bar code” or array configurations.

In smart hologram sensors (Smart Holograms Ltd, Cambridge, UK), a holographic image is stored in a thin polymer film that is sensitised to react specifically with the biomarker. On exposure, the biomarker reacts with the polymer leading to an alteration in the image displayed by the hologram. The test result read-out can be a change in the optical brightness, image, colour and/or position of the image. For qualitative and semi-quantitative applications, a sensor hologram can be read by eye, thus removing the need for detection equipment. A simple colour sensor can be used to read the signal when quantitative measurements are required. Opacity or colour of the sample does not interfere with operation of the sensor. The format of the sensor allows multiplexing for simultaneous detection of several substances. Reversible and irreversible sensors can be designed to meet different requirements, and continuous monitoring of a particular biomarker of interest is feasible.

Suitably, biosensors for detection of the biomarker of the invention are coupled, i.e. they combine biomolecular recognition with appropriate means to convert detection of the presence, or quantitation, of the biomarker in the sample into a signal. Biosensors can be adapted for “alternate site” diagnostic testing, e.g. in the ward, outpatients' department, surgery, home, field and workplace.

Biosensors to detect the biomarker of the invention include acoustic, plasmon resonance, holographic and microengineered sensors. Imprinted recognition elements, thin film transistor technology, magnetic acoustic resonator devices and other novel acousto-electrical systems may be employed in biosensors for detection of the biomarkers of the invention.

Methods involving detection and/or quantification of the biomarker of the invention can be performed using bench-top instruments, or can be incorporated onto disposable, diagnostic or monitoring platforms that can be used in a non-laboratory environment, e.g. in the physician's office or at the patient's bedside. Suitable sensors or biosensors for performing methods of the invention include “credit” cards with optical or acoustic readers. Sensors or biosensors can be configured to allow the data collected to be electronically transmitted to the physician for interpretation and thus can form the basis for e-neuromedicine.

A higher level of the VLDL and/or LDL biomarkers in the test biological sample relative to the level in a normal control is indicative of the presence of a psychotic disorder, in particular a schizophrenic disorder, bipolar disorder, or predisposition thereto.

The invention also comprises detecting and/or quantifying a transthyretin peptide biomarker, preferably comprising the amino acid sequence of SEQ ID NO: 1, or a fragment thereof, in a test biological sample from a test subject and comparing the level of peptide present in said test sample with one or more controls.

The invention further comprises detecting and/or quantifying an ApoA1 peptide biomarker comprising the amino acid sequence of SEQ ID NO: 2, or a fragment thereof, in a test biological sample from a test subject and comparing the level of peptide present in said test sample with one or more controls.

The control used in a method of the invention can be one or more controls selected from the group consisting of: the level of biomarker found in a normal control sample from a normal subject, a normal biomarker level or a normal biomarker concentration range.

Suitably, the test and the normal control sample will be the same type of sample, e.g. the level in a test serum sample will be compared with the level in a control serum sample.

A preferred method of diagnosing a schizophrenic disorder or predisposition thereto, comprises:

(a) quantifying the amount of a peptide biomarker comprising SEQ ID NO: 1 or 2, or a fragment thereof present in a test biological sample, and
(b) comparing the amount of said peptide in said test sample with the amount present in a normal control biological sample from a normal subject.

A lower level of the transthyretin peptide biomarker in the test sample relative to the level in the normal control is indicative of the presence of a schizophrenic disorder or predisposition thereto. An equivalent or higher level of said peptide in the test sample relative to the normal control is indicative of absence of a schizophrenic disorder and/or absence of a predisposition thereto.

Efficient diagnosis and monitoring methods provide very powerful “patient solutions” with the potential for improved prognosis, by establishing the correct diagnosis, allowing rapid identification of the most appropriate treatment (thus lessening unnecessary exposure to harmful drug side effects), reducing “down-time” and relapse rates.

Also provided is a method of monitoring efficacy of a therapy for a schizophrenic disorder in a subject having such a disorder, suspected of having such a disorder or of being predisposed thereto, comprising detecting and/or quantifying a transthyretin peptide, preferably comprising the amino acid sequence of SEQ ID NO: 1, or a fragment thereof, present in a biological sample from said subject. In monitoring methods, test samples may be taken on two or more occasions. The method may further comprise comparing the level of the biomarker present in the test sample with one or more controls and/or with one or more previous test samples taken earlier from the same test subject, e.g. prior to commencement of therapy, and/or from the same test subject at an earlier stage of therapy. The method may comprise detecting a change in the level of the biomarker in test samples taken on different occasions.

The invention provides a method for monitoring efficacy of therapy for a schizophrenic disorder in a subject, comprising:

(a) quantifying the amount of a transthyretin peptide biomarker, preferably comprising the amino acid sequence of SEQ ID NO: 1 or a fragment thereof, in a test biological sample taken from said subject, and
(b) comparing the amount of said peptide in said test sample with the amount present in one or more controls and/or one or more previous test samples taken at an earlier time from said same test subject.

An increase in the level of the peptide biomarker in the test sample relative to the level in a previous test sample taken earlier from the same test subject is indicative of a beneficial effect, e.g. stabilisation or improvement, of said therapy on the disorder, suspected disorder or predisposition thereto.

Methods for monitoring efficacy of a therapy can be used to monitor the therapeutic effectiveness of existing therapies and new therapies in human subjects and in non-human animals (e.g. in animal models). These monitoring methods can be incorporated into screens for new drug substances and combinations of substances.

Suitably, the time elapsed between taking samples from a subject undergoing diagnosis or monitoring will be 3 days, 5 days, a week, two weeks, a month, 2 months, 3 months, 6 or 12 months. Samples may be taken prior to and/or during and/or following an anti-schizophrenic disorder therapy. Samples can be taken at intervals over the remaining life, or a part thereof, of a subject.

Quantifying the amount of the biomarker present in a sample may include determining the concentration of the peptide biomarker present in the sample. Detecting and/or quantifying may be performed directly on the sample, or indirectly on an extract therefrom, or on a dilution thereof.

Detecting and/or quantifying can be performed by any method suitable to identify the presence and/or amount of a specific protein in a biological sample from a patient or a purification of extract of a biological sample or a dilution thereof. In methods of the invention, quantifying may be performed by measuring the concentration of the peptide biomarker in the sample or samples. Biological samples that may be tested in a method of the invention include cerebrospinal fluid (CSF), whole blood, blood serum, urine, saliva, or other bodily fluid (stool, tear fluid, synovial fluid, sputum), breath, e.g. as condensed breath, or an extract or purification therefrom, or dilution thereof. Biological samples also include tissue homogenates, tissue sections and biopsy specimens from a live subject, or taken post-mortem. Preferably, the sample is CSF or blood serum. The samples can be prepared, for example where appropriate diluted or concentrated, and stored in the usual manner.

Detection and/or quantification of transthyretin peptide biomarkers may be performed by detection of the peptide biomarker or of a fragment thereof, e.g. a fragment with C-terminal truncation, and/or with N-terminal truncation. Fragments are suitably greater than 4 amino acids in length. Preferably, fragments are in the range of from about 6 to about 50 amino acids in length.

The biomarker may be directly detected, e.g. by SELDI or MALDI-TOF. Alternatively, the biomarker may be detected, directly or indirectly, via interaction with a ligand or ligands such as an antibody or a biomarker-binding fragment thereof, or other peptide, or ligand, e.g. aptamer, or oligonucleotide, capable of specifically binding the biomarker. The ligand may possess a detectable label, such as a luminescent, fluorescent or radioactive label, and/or an affinity tag. Ligands include, for example:

(1) in vivo: T3, T4 (thyroid hormones), vitamin A (indirectly by interacting with serum retinol-binding protein), apolipoprotein AI (ApoAI), noradrenaline oxidation products, and pterins.
(2) in vitro (most of them pharmacological agents): some non-steroidal anti-inflammatory drugs (NSAIDs), environmental pollutants, such as polyhalogenated biphenyls and thyromimetic compounds, xanthone derivatives as well as natural and synthetic flavonoids. Other ligands may be antibodies as listed in Table 1.

For example, methods relating to detecting, monitoring, diagnosing and/or quantifying can be performed by one or more methods selected from the group consisting of: SELDI (-TOF), MALDI (-TOF), a 1-D gel-based analysis, a 2-D gel-based analysis, Mass spec (MS), LC and LC-MS-based techniques. Appropriate LC MS techniques include ICAT® (Applied Biosystems, CA, USA), or iTRAQ® (Applied Biosystems, CA, USA). Liquid chromatography (e.g. high pressure liquid chromatography (HPLC) or low pressure liquid chromatography (LPLC)), thin-layer chromatography, NMR (nuclear magnetic resonance) spectroscopy could also be used.

Methods for diagnosis or monitoring according to the invention may comprise analysing a biological sample, e.g. cerebrospinal fluid (CSF) or serum, by SELDI TOF, MALDI TOF and other methods using mass spectrometry to detect the presence or level of the peptide biomarker comprising SEQ ID NO: 1 or 2 or a fragment thereof. Such techniques may be used for relative and absolute quantification and also to assess the ratio of the biomarker according to the invention with other biomarkers that may be present. These methods are also suitable for clinical screening, prognosis, monitoring the results of therapy, identifying patients most likely to respond to a particular therapeutic treatment, for drug screening and development, and identification of new targets for drug treatment.

Surface enhanced laser deionization ionization (SELDI) mass spectrometry is a powerful tool for identifying a characteristic “fingerprint” of proteins and peptides in body fluids and tissues for a given condition, e.g. drug treatments and diseases¹⁹. This technology utilizes protein chips to capture proteins/peptides and a time-of-flight mass spectrometer (tof-MS) to quantitate and calculate the mass of compounds ranging from small molecules and peptides of less than 1,000 Da up to proteins of 500 kDa. Quantifiable differences in protein/peptide patterns can be statistically evaluated using automated computer programs which represent each protein/peptide measured in the biofluid spectrum as a coordinate in multi-dimensional space. This approach has been most successful in the field of clinical biomarker discovery as it can be used as a diagnostic tool without knowing the biomarkers' identity. The SELDI system also has a capability of running hundreds of samples in a single experiment. In addition, all the signals from SELDI mass spectrometry are derived from native proteins/peptides (unlike some other proteomics technologies which require protease digestion), thus directly reflecting the underlying physiology of a given condition.

Detecting and/or quantifying the transthyretin peptide biomarker may be performed using any method based on immunological, peptide, aptamer or synthetic recognition. For example, the method may involve an antibody, or a fragment thereof capable of specific binding to the transthyretin peptide biomarker, e.g. to a peptide comprising or consisting of the amino acid sequence shown in SEQ ID NO: 1 or 2 or a fragment thereof. Suitable antibodies that bind human TTR are commercially available, and are listed in Table 1.

TABLE 1
Goat Anti-Prealbumin Polyclonal Antibody, Unconjugated
Abcam
Rabbit Anti-Prealbumin Polyclonal Antibody, Unconjugated
Abcam
Rabbit Anti-Prealbumin Polyclonal Antibody, Unconjugated
Abcam
Sheep Anti-Prealbumin Polyclonal Antibody, Unconjugated
Abcam
Mouse Anti-Human TTR Monoclonal antibody, Unconjugated, 4D8
Abnova Corporation
Goat Anti-Prealbumin Polyclonal Antibody, Unconjugated
BIODESIGN International
Sheep Anti-Albumin, pre Polyclonal Antibody, Alkaline Phosphatase
Conjugated
Biogenesis
Sheep Anti-Human Albumin, pre Polyclonal Antibody, FITC Conjugated
Biogenesis
Sheep Anti-Human Albumin, pre Polyclonal Antibody,
Horseradish Peroxidase
Conjugated
Biogenesis
Sheep Anti-Human Albumin, pre Polyclonal Antibody, Unconjugated
Biogenesis
Goat Anti-Prealbumin Polyclonal Antibody, Unconjugated
Fitzgerald Industries International
Goat Anti-Prealbumin Polyclonal Antibody, Unconjugated
GeneTex
Rabbit Anti-Prealbumin Polyclonal Antibody, Unconjugated
GeneTex
Sheep Anti-Prealbumin Polyclonal Antibody, Unconjugated
GeneTex
Goat Anti-Prealbumin Polyclonal Antibody, Unconjugated
Novus Biologicals
Rabbit Anti-Prealbumin Polyclonal Antibody, Unconjugated
Novus Biologicals
Sheep Anti-Prealbumin Polyclonal Antibody, Unconjugated
Novus Biologicals
Goat Anti-Prealbumin Polyclonal Antibody, Unconjugated
Sigma-Aldrich
Anti-Human Prealbumin, Delipidated Polyclonal Antibody,
Unconjugated
United States Biological
Anti-Human Retinol Binding Protein Monoclonal Antibody,
Unconjugated,
Clone 0.N.549
United States Biological
Rabbit anti-human
Dako

Suitable immunological methods include sandwich immunoassays, such as sandwich ELISA in which the detection of the peptide biomarkers is performed using two antibodies which recognize different epitopes on the peptide biomarker (see examples); radioimmunoassays (RIA), direct or competitive enzyme-linked immunosorbent assays (ELISA), enzyme immunoassays (EIA), western blotting, immunoprecipitation and any particle-based immunoassay (e.g. using gold, silver, or latex particles, magnetic particles, or Q-dots). Immunological methods may be performed, for example, in microtitre plate or strip format.

In methods and uses of the invention in which the amount of the transthyretin biomarker peptide of SEQ ID NO: 1 or a fragment thereof present in a test sample from a test subject is measured, detection of a lower level of the biomarker peptide in the test sample compared to the level found in a normal control sample from a normal individual is indicative of a schizophrenic disorder or a predisposition thereto in the test subject. For example in serum, the amount of transthyretin peptide of SEQ ID NO: 1, a fragment or derivative thereof, detected in a sample from a test subject with a schizophrenic disorder or predisposition thereto will generally be at least 15% lower than the amount of the transthyretin peptide found in a normal control sample. In the prefrontal cortex, the decrease of transthyretin expression is about 40%. In CSF samples, the decrease is about 20% compared to TTR levels in normal control samples.

According to the invention, it is also possible to assess, for example using mass spectrometry or other suitable techniques, a decrease of the TTR peptide of SEQ ID No 1 or a fragment thereof by reference to the levels of a control peptide or protein. Such peptide may be another biomarker for a schizophrenic disorder.

In methods and uses of the invention in which the amount, level or concentration of the ApoA1 biomarker peptide of SEQ ID NO: 2 or a fragment thereof present in a test sample from a test subject is measured, detection of a lower level of the biomarker peptide in the test sample compared to the level found in a normal control sample from a normal individual is indicative of a schizophrenic disorder or a predisposition thereto in the test subject. For example, the level of peptide of SEQ ID NO: 2 detected in a test sample from a test drug-naïve subject with a schizophrenic disorder or predisposition thereto will generally be at least about 10% to about 80%, preferably about 18% to about 60%, lower than the amount of the peptide found in a normal control sample.

Biological samples that may be tested in a method of the invention include cerebrospinal fluid (CSF), whole blood, blood serum, plasma, red blood cells, liver cells, urine, saliva, or other tissue or bodily fluid (stool, tear fluid, synovial fluid, sputum), breath, e.g. as condensed breath, or an extract or purification therefrom, or dilution thereof. Biological samples also include tissue homogenates, tissue sections and biopsy specimens from a live subject, or taken post-mortem. The samples can be prepared, for example where appropriate diluted or concentrated, and stored in the usual manner.

In a serum sample from a test drug-naïve subject with a schizophrenic disorder or predisposition thereto, the level of the ApoA1 biomarker peptide comprising SEQ ID NO: 2 or a fragment thereof detected and/or quantified according to the methods of the invention will be about 10% to about 25% lower, preferably about 18% lower, than the amount of the peptide found in a normal control serum sample.

In a red blood cells sample from a test drug-naïve subject with a schizophrenic disorder or predisposition thereto, the level of the ApoA1 biomarker peptide comprising SEQ ID NO: 2 or a fragment thereof detected and/or quantified according to the methods of the invention will preferably be about 50% to about 70% lower, preferably about 60% lower, than the amount of the peptide found in a normal control red blood cell sample.

In liver cells from a test drug-naïve subject with a schizophrenic disorder or predisposition thereto, the level of ApoA1 peptide biomarker peptide comprising SEQ ID NO: 2 or a fragment thereof detected and/or quantified according to the methods of the invention will preferably be about 20% to about 40% lower, preferably about 30% lower, than the amount of the peptide found in a normal control liver cell sample.

In a CSF sample from a test drug-naïve subject with a schizophrenic disorder or predisposition thereto, the level of ApoA1 peptide biomarker peptide comprising SEQ ID NO: 2 or a fragment thereof detected and/or quantified according to the methods of the invention will preferably be about 20% to about 40% lower, preferably about 30% to about 35% lower, than the amount of the peptide found in a normal control CSF sample.

Detection and/or quantification of ApoA1 peptide biomarkers may be performed by detection of the peptide biomarker or of a fragment thereof, e.g. a fragment with C-terminal truncation, or with N-terminal truncation. Fragments are suitably greater than 4 amino acids in length.

The biomarker may be directly detected, e.g. by SELDI, MALDI-TOF. Alternatively, the biomarker may be detected directly or indirectly via interaction with any naturally occurring, biologically derived or synthetic ligand or ligands such as an antibody or a biomarker-binding fragment thereof, or other peptide, or ligand, e.g. aptamer, or oligonucleotide, or chemically-synthesised binding partner, capable of specifically binding the biomarker. Ligands used in the methods of the invention may possess a detectable label, such as a luminescent, coloured, metallic, magnetic, fluorescent or radioactive label, and/or an affinity tag (e.g. Arg-tag, calmodulin-binding peptide, cellulose-binding domain, DsbA, c-myc-tag, glutathione S-transferase, FLAG-tag, HAT-tag, His-tag, maltose-binding protein, NusA, S-tag, SBP-tag, Strep-tag, or thioredoxin). The marker may also comprise nanoparticles. Quantum dots Qdots) may be used. Qdots are core/shell CdSe/ZnS nanocrystals of a few nanometers in size, which can be conjugated to biomolecules.

The ligand may also be labelled with up-converting phosphors. Up-converting phosphors are uniform submicron up-converting phosphors microspheres that can be synthesised and coated with biologically active probes, such as antibodies. They are materials that emit visible light upon excitation with near infra-red light.

Depending on the nature of the ligand, Fluorescence Resonance Energy Transfer (FRET), channeling assays (e.g. Luminescent oxygen channeling, for example using LOCI® latex particles conjugated to the biomolecule) or proximity assays may be used for detection.

Furthermore, surface plasmon resonance (SPR) may be used as a label-free sensing tool.

Detecting and/or quantifying can be performed by one or more methods selected from the group consisting of: LC, HPLC, CZE, SELDI (-TOF), MALDI (-TOF), a 1-D gel-based analysis, a 2-D gel-based analysis, e.g. Differential In Gel Electrophoresis (2D-DIGE), Mass spec (MS) and LC-MS-based techniques. Appropriate LC MS techniques include ICAT® (Applied Biosystems, CA, USA), or iTRAQ® (Applied Biosystems, CA, USA). Liquid chromatography (e.g. high pressure liquid chromatography (HPLC) or low pressure liquid chromatography (LPLC)), thin-layer chromatography, NMR (nuclear magnetic resonance) spectroscopy could also be used.

Methods for diagnosis according to the invention may comprise analysing a biological sample, e.g. cerebrospinal fluid (CSF), serum or plasma, by SELDI TOF or MALDI TOF to detect the presence or level of the peptide biomarker of SEQ ID NO: 2 or a fragment thereof.

Detecting and/or quantifying the ApoA1 peptide biomarker may be performed using an immunological method, involving an antibody, or a fragment thereof capable of specific binding to the ApoA1 peptide biomarker, e.g. an antibody to a peptide consisting of the amino acid sequence shown in SEQ ID NO: 2 or a fragment thereof. Suitable immunological methods include sandwich immunoassays, such as sandwich ELISA in which the detection of the peptide biomarkers is performed using two, antibodies which recognize different epitopes on the peptide biomarker; radioimmunoassays (RIA), direct or competitive enzyme linked immunosorbent assays (ELISA) or any modification or embodiment thereof, enzyme-immuno assays (EIA), western blotting, immunoprecipitation and any particle-based immunoassay (e.g. using gold, silver, or latex particles, magnetic particles, or Q-dots). Immunological methods may be performed, for example, in microtitre plate or strip format.

Biosensors according to the invention may comprise a ligand or ligands, as described herein, capable of specific binding to the peptide biomarker. Such biosensors are useful in detecting and/or quantifying a peptide of the invention.

Also provided is an array, pattern or signature comprising a ligand as described herein capable of specific binding to a peptide biomarker.

Diagnostic or monitoring kits are provided for performing methods of the invention. Such kits will suitably comprise a ligand as described herein, for detection and/or quantification of the peptide biomarker, and/or a biosensor, and/or an array as described herein, optionally together with instructions for use of the kit.

Also provided by the invention is the use of a ligand as described herein, which may be naturally occurring or chemically synthesised, and is suitably a peptide, antibody or fragment thereof, aptamer or oligonucleotide, or the use of a biosensor of the invention, or an array of the invention, or a kit of the invention to detect and/or quantify the peptide biomarker or a fragment thereof. In this use, the detection and/or quantification can be performed on a biological sample, such as CSF, whole blood, blood serum, tear fluid, urine, saliva, or other bodily fluid, breath, e.g. as condensed breath, or an extract or purification therefrom, or dilution thereof.

Thus, in a further aspect of the invention, there is provided the use of a ligand, as described herein, which can be a peptide, antibody or fragment thereof or aptamer or oligonucleotide according to the invention; or the use of a biosensor according to the invention, or an array according to the invention; or a kit according to the invention, to identify a substance capable of stimulating, promoting or activating the generation of a peptide biomarker.

Also there is provided a method of identifying a substance capable of stimulating, promoting or activating the generation of a peptide biomarker, the peptide biomarker preferably comprising the amino acid sequence of SEQ ID NO: 1 or 2, or a fragment thereof, in a subject, comprising administering a test substance to a subject animal and detecting and/or quantifying levels of the peptide biomarker present in a test sample from the subject.

Any suitable animal may be used as a subject non-human animal, for example a non-human primate, horse, cow, pig, goat, zebrafish, sheep, dog, cat, fish, rodent, e.g. guinea pig, rat or mouse; insect (e.g. Drosophila), amphibian (e.g. Xenopus) or C. elegans.

The test substance can be a known chemical or pharmaceutical substance, such as, but not limited to, an anti-schizophrenic disorder therapeutic, or the test substance can be a novel synthetic or natural chemical entity, or a combination of two or more of the aforesaid substances.

There is provided a method of identifying a substance capable of stimulating, promoting or activating the generation of a peptide biomarker, preferably comprising the amino acid sequence of SEQ ID NO: 1 or 2, or a fragment thereof, in a subject, comprising exposing a test cell to a test substance and monitoring levels of the peptide biomarker within said test cell, or secreted by said test cell. The test cell could be prokaryotic, however it is preferred that a eukaryotic cell be employed in cell-based testing methods. Suitably, the eukaryotic cell is a yeast cell, insect cell, Drosophila cell, amphibian cell (e.g. from Xenopus), C. elegans cell or is a cell of human, non-human primate, equine, bovine, porcine, caprine, ovine, canine, feline, piscine, rodent or murine origin.

In methods for identifying substances of potential therapeutic use, non-human animals or cells can be used that are capable of expressing human transthyretin polypeptides.

Screening methods also encompass a method of identifying a ligand capable of binding to a peptide biomarker according to the invention, comprising incubating a test substance in the presence of the peptide biomarker in conditions appropriate for binding, and detecting and/or quantifying binding of the peptide to said test substance.

High-throughput screening technologies based on the biomarkers, uses and methods of the invention, e.g. configured in an array, pattern or signature format, are suitable to monitor biomarker signatures for the identification of potentially useful therapeutic compounds, e.g. ligands such as natural compounds, synthetic chemical compounds (e.g. from combinatorial libraries), peptides, monoclonal or polyclonal-antibodies or fragments thereof, capable of binding the biomarker.

Methods of the invention can be performed in array, pattern or signature format, e.g. on a chip, or as a multiwell array. As described above, other techniques, such as mass spectrometry can also be used. Methods can be adapted into platforms for single tests, or multiple identical or multiple non-identical tests, and can be performed in high throughput format. Methods of the invention may comprise performing one or more additional, different tests to confirm or exclude diagnosis, and/or to further characterise a condition.

The invention further provides a substance, e.g. a ligand, identified or identifiable by an identification or screening method or use of the invention. Such substances may be capable of stimulating, promoting or activating, directly or indirectly, the activity of a peptide biomarker, or of stimulating, promoting or activating generation of the peptide biomarker. The term substances includes substances that do not directly bind the peptide biomarker and directly induce expression of the peptide biomarker or promote or activate a function, but instead indirectly induce expression of the peptide biomarker or promote/activate a function of the peptide biomarker. Ligands are also included in the term substances; ligands of the invention (e.g. a natural or synthetic chemical compound, peptide, aptamer, oligonucleotide, antibody or antibody fragment) are capable of binding, preferably specific binding, to a peptide biomarker.

The invention further provides the use of a substance or ligand according to the invention in the treatment of a schizophrenic disorder or predisposition thereto.

Also provided is the use of a substance according to the invention as a medicament.

Yet further provided is the use of a substance according to the invention in the manufacture of a medicament for the treatment of a schizophrenic disorder or predisposition thereto.

A kit for diagnosing or monitoring a schizophrenic disorder or predisposition thereto is provided. Suitably a kit according to the invention may contain one or more components selected from the group: a ligand specific for a peptide biomarker, a peptide biomarker, a control, a reagent, and a consumable; optionally together with instructions for use of the kit.

The terms “treating” or “treatment” as used herein with reference to therapeutic uses of the biomarker of the invention describe the management or care of a patient for the purposes of combating disease, and include the administration of the active agents to asymptomatic individuals, for example to prevent the onset of the symptoms or complications (i.e. prophylaxis).

Also, there is provided a method for identifying a schizophrenic disorder therapeutic substance, wherein said substance is capable of promoting the generation of an ApoA1 peptide biomarker, said method comprising administering said substance to a test subject, and detecting and/or quantifying the level of ApoA1 peptide biomarker in said test subject. In another embodiment, there is provided a method for identifying a schizophrenic disorder therapeutic substance wherein said substance is capable of promoting the activity of an ApoA1 peptide biomarker, said method comprising administering said substance to a test subject, and detecting and/or quantifying the activity of ApoA1 peptide biomarker in said test subject. An increase in the level or activity of an ApoA1 biomarker peptide indicates that the substance is schizophrenic disorder therapeutic substance. Preferably, the ApoA1 peptide biomarker according to these methods comprises SEQ ID NO:2, a fragment thereof or a non-human ApoA1 homolog.

The term “therapeutic substance” as used herein defines a substance that has therapeutic, i.e. curative/beneficial properties and treats a schizophrenic disorder, alleviates the symptoms thereof or prevents the onset of a schizophrenic disorder. Thus, the substance is for use in the treatment of schizophrenia.

The said test subject according to a method for identifying a schizophrenia disorder therapeutic substance may be any suitable animal, preferably a non-human animal, for example a non-human primate, horse, cow, pig, goat, sheep, dog, cat, fish, rodent, e.g. guinea pig, rabbit, rat or mouse; insect (e.g. Drosophila), amphibian (e.g. Xenopus) or C. elegans.

The test substance can be a known chemical or pharmaceutical substance, such as, but not limited to, an anti-schizophrenic therapeutic or a known anti-psychotic; or the test substance can be novel synthetic or natural chemical entity, or a combination of two or more of the aforesaid substances.

The invention further provides an in vitro method of identifying a schizophrenic disorder therapeutic substance wherein said substance is capable of stimulating or promoting the generation of an ApoA1 peptide biomarker, said method comprising exposing a test cell to a test substance and detecting an increased level of said biomarker peptide or a fragment thereof within said test cell or secreted by said test cell. Also provided is an in vitro method of identifying a schizophrenic disorder therapeutic substance wherein said substance is capable of stimulating or promoting the activity of an ApoA1 peptide biomarker, said method comprising exposing a test cell to a test substance and detecting an increased activity of said biomarker peptide or a fragment thereof within said test cell or secreted by said test cell. Preferably, the ApoA1 peptide biomarker according to these in vitro methods comprises SEQ ID NO:2, a fragment thereof or a non-human ApoA1 homolog.

Suitably, the eukaryotic cell can be selected from a yeast cell, insect cell, Drosophila cell, amphibian cell (e.g. from Xenopus), C. elegans cell or the cell can be of human, non-human primate, equine, bovine, leporine, porcine, caprine, ovine, canine, feline, piscine, rodent or murine origin.

In methods for identifying substances of potential therapeutic use, non-human animals or cells can be used that are capable of expressing human ApoA1 polypeptides. Alternatively, the non-human cells may express their endogenous ApoA1.

Screening methods also encompass a method of identifying a ligand capable of binding to an ApoA1 peptide biomarker according to the invention, comprising incubating a test substance in the presence of the peptide biomarker in conditions appropriate for binding, and detecting and/or quantifying binding of the peptide to said test substance.

Also provided is a substance identified by a method according to the invention.

Diagnostic or monitoring kits are provided for performing methods of the invention. Such kits will suitably comprise a ligand as described herein capable of specific binding to the ApoA1 peptide biomarker, for detection and/or quantification of the ApoA1 peptide biomarker, and/or a biosensor, and/or an array as described herein, optionally together with instructions for use of the kit. In another aspect, the invention provides a kit for diagnosing or monitoring a schizophrenic disorder or predisposition thereto. Suitably, a kit according to the invention may contain one or more components selected from the group: a ligand specific for an ApoA1 peptide biomarker, an ApoA1 peptide biomarker or a structural/shape mimic of an ApoA1 peptide biomarker, a control, a reagent, and a consumable; optionally together with instructions for use of the kit.

Methods of the invention can be performed in multi-analyte panel or array format, e.g. on a chip, or as a multiwell array. Methods can be adapted into platforms for single tests, or multiple identical or multiple non-identical tests, and can be performed in high throughput format. Methods of the invention may comprise performing one or more additional, different tests to confirm or exclude diagnosis, and/or to further characterise a psychotic condition.

### LIST OF FIGURES

FIG. 1 Metabonomic analysis of plasma samples from monozygotic twins discordant for schizophrenia and control twins. (A) Partial ¹H NMR spectrum of plasma samples from a pair of representative twins discordant with schizophrenia (the affected co-twin in grey and the unaffected in black) illustrate changes in lipid regions—(CH₂)ₙ and CH₃ lipids. (B) and (C) PLS-DA scores plots showing differentiation of control twin from unaffected and affected twins with schizophrenia as determined by the ¹H NMR plasma spectra. The unaffected co-twin shows an intermediate position between controls and the schizophrenic co-twin.

FIG. 2 Metabonomic analysis of plasma samples from female twins discordant for schizophrenia and female control twins. PLS-DA scores plots (FIG. 2A) of female monozygotic twins showing a clear differentiation of control twins, unaffected twins and the schizophrenic twins as determined by the ¹H NMR plasma spectra. The loading plots demonstrate that LDL (0.86 and 1.26), VLDL (0.9 and 1.3) and aromatic regions (˜7.5) are the key chemical shifts that contribute to the separation. There is a high degree of similarity between FIG. 1C and FIG. 2B.

FIG. 3 Negative correlations between global functioning score (DSM IV, Axis V) and two key chemical shifts (1.24-1.28 ppm; A and 1.28-1.32 ppm; B) primarily corresponding to LDL and VLDL levels in female twin plasma. The R² are shown in each plots.

FIG. 4 Metabonomic analysis of plasma samples from male discordant twins with and without schizophrenia and male control twins. PLS-DA scores plots (A) showing no differentiation between male control twins and unaffected male twins whilst schizophrenic twins show a moderate differentiation from male control twins and male discordant twins. Glucose level (3.2-3.9 ppm) and signals from aromatic region (˜7.9 ppm) and 1.04-1.06-1.12 ppm regions were found to be the major contributing factor for separation as illustrated in the loading plots (B).

FIG. 5 Protein/peptide profiling of CSF samples from first-onset, drug-naïve schizophrenia patients using SELDI mass spectrometry. A: A typical CSF protein/peptide spectrum using an anion exchanger chip (Q10; 50 mM Tris-HCl, pH9.0) showing the m/z range of about 10,000-15,000 from a healthy volunteer.

B: The peak intensity of protein/peptide peaks from SELDI spectra were analyzed using PCA and PLS-DA models. A 3D PLS-DA scores plot indicates clusters of healthy volunteers (in black) and untreated, drug-naïve schizophrenia patients (in grey).
C: and D PLS-DA scores and loadings plots. The scores plot is similar to (B) but only the first two components were used to discriminate healthy controls and patients. The loading plot as shown in (D) indicates the key protein/peptide peaks contributing the most towards the separation in (C).

FIG. 6 Down-regulation of three different forms of transthyretin in CSF from first onset, drug-naïve schizophrenia patients. Examples of CSF spectra from healthy volunteers and patients with schizophrenia within 6-17 kDa are shown in (A). The peak cluster indicated (arrow) is enlarged in (B). Statistical details of each sub-peak are listed in the table below. On-chip reduction of CSF peptide/protein with β-mecaptoethanol at room temperature showed that the three peaks 13,741, 13,875, and 13,923 Da were reduced into a single peak (C), suggesting they are different S-cysteinylated derivatives of the same protein. To identify the identity, CSF samples from a healthy volunteer and a schizophrenia patient were applied to an anion exchanger column (HyperD) and eluted with pH9-pH3 buffers. A major band was eluted at ˜14-15 kDa in pH3 fraction (D, left panel). The band was identified as transthyretin using LC-MS/MS (D, right panel) and the sequence coverage is shown. In addition, the band was confirmed to be the peak cluster around 13.5-14 kDa in the spectra by eluting the proteins from the band and running on a NP20 chip to match the mass (E).

FIG. 7 Transthyretin levels in sera of first onset, drug-naïve schizophrenia patients and prefrontal cortex post-mortem tissue from schizophrenia patients. The serum samples from the same patients whose CSF protein profiles were measured in FIGS. 5 and 8 were included in this study. FIG. 7A shows serum transthyretin levels in schizophrenia patients significantly decreased by ˜15% compared to control subjects. Data are shown in Mean±S.D. *p=0.007 (t test). FIG. 7B indicates no correlation between serum transthyretin and CSF SELDI signals from one of the transthyretin isoforms (m/z=13,741) in the 2^{nd} sample set (for demographics, see table 5). Similar results were found when comparing with signals from other isoforms in CSF (data not shown). FIG. 7C shows a ˜40% decrease of transthyretin expression in prefrontal cortex of 5 schizophrenia patients and 5 control subjects. For demographic details, see Table 6.

FIG. 8 PLS-DA analysis of CSF protein/peptide profiles from an independent validation sample set containing 18 first-onset, drug-naïve schizophrenia patients and 40 healthy volunteers. The demographic details of this sample set are listed in Table 5. The PLS-DA scores plot showed a separation between patients (black) and healthy volunteers (grey). The loadings plot indicates transthyretin protein signals between 13,600 and 14,000 found in the first experiment (see FIG. 5).

FIG. 9. Down-regulation of CSF ApoA1 levels in first-onset drug-naïve schizophrenia patients. A: Strong anion-exchange (Q10) ProteinChip arrays were used to profile CSF proteins and peptides. A representative CSF SELDI spectrum showing the profile of proteins/peptides with a mass-to-charge ratio between m/z=4,000 to 70,000 m/z. B: a gel view of 14 representative spectra showing apoA1 protein (˜28 kDa) to be reduced (−35%; p=0.00001) in schizophrenia samples compared to controls. The adjacent histogram depicts the mean+/−SD of ApoA1 levels (41 CSF samples from first-onset drug-naïve schizophrenia patients were compared to 40 matched control samples). C: The ˜28 kDa peak was gel purified (arrow) and the excised protein was sequenced using LC-MS/MS (right panel). The sequence coverage that is highlighted in bold corresponds to part of the published ApoA1 amino acid sequence. D: the band was confirmed to be the peak cluster around 28 kDa in the spectra by immuno-capturing the proteins in CSF samples with an anti-ApoA1 antibody on-chip (RS-100 ProteinChip). CSF samples were applied to RS-100 chips coupled with (lower panel) or without (upper panel) anti-ApoA1 antibody. The proteins bound to the chips were analyzed by SELDI-TOF. The captured ApoA1 protein shows an m/z at 28 kDa. E: Western blot analysis showing ApoA1 expression in the prefrontal cortex of 8 schizophrenia patients and 8 healthy volunteers. A trend towards down-regulation was observed (−32%; p=0.07, t test). The protein loadings are shown below the blot.

FIG. 10. 2-D DIGE analysis of liver from schizophrenia patients (n=15) and controls (n=15). A: A typical 2D gel image of liver protein extracts. ApoA1 protein (the corresponding spot is indicated by an arrow) was one of the significantly altered proteins. B: ApoA1 levels were found to be significantly reduced in livers from schizophrenia patients (−30%; p=0.017). The histogram depicts the mean+/−SD of the relative standardised abundance. C: LC-MS/MS analysis of trypsinized peptides from the gel spot showed that three peptide fragments were derived from apoA1 protein. The Mascot score and sequence coverage are shown in the table.

FIG. 11 2-D DIGE analysis of red blood cells (RBC), from schizophrenia patients (n=20) and controls (n=20), illustrating a decrease in ApoA1 protein expression. A: A typical 2D gel image of the unfractionated RBC proteome, showing a dominant expression of haemoglobin proteins (pI˜7). B: A typical 2D gel image after removing dominant proteins (i.e. haemoglobin) by a Ficoll density gradient. The arrow and spot indicates the position the apoA1 spot on the gel. C: ApoA1 levels were found to be significantly reduced in RBC from schizophrenia patients (−60%; p=0.0034). The histogram depicts the mean+/−SD of the relative standardised abundance. D: LC-MS/MS analysis of trypsinized peptides from the gel spot identified that eight peptide fragments were derived from ApoA1 protein. The Mascot score and sequence coverage are shown in the table.

FIG. 12 Down-regulation of serum ApoA1 levels in schizophrenia. A: ELISA analysis of apoA1 levels in sera of first-onset drug-naïve schizophrenia patients (n=35) and healthy volunteers (n=63). The mean value+/−S.D. of apoA1 concentrations in schizophrenia patients and controls is shown. p=0.00039 (t test). B: Correlation analysis of CSF and serum ApoA1 levels from the same individuals. No correlation was found between serum and CSF levels for either the control or patient group.

FIG. 13 illustrates that a high sensitivity of about 89% and a specificity of about 73% can be achieved when combining 2 biomarkers for PCA analysis.

### EXAMPLES

The invention will be further understood by reference to the Examples provided below.

### Example 1

Plasma samples from 21 pairs of monozygotic twins discordant for schizophrenia and 16 matched control twins were collected under standardised conditions by Dr Fuller Torrey, Stanley Medical Research Institute, Bethesda, USA. All study participants gave their written informed consent and the original study was approved by an Institutional Review Board. The GAF of each individual was derived by consensus of the two interviewers who did the SCID interview Structured Clinical Interview for DSM-IV-TR (SCID). SCID is a clinical rating scale which involves a semi-structured diagnostic interview designed to assist clinicians, researchers, and trainees in making reliable DSM-IV psychiatric diagnoses. The plasma was obtained from both twins simultaneously as part of a lymphocyte collection aphoresis procedure carried out at mid-morning, with both twins having been on similar diets and residing in a hotel together. Blood plasma samples (50 μl) were made up to a final volume of 500 μl by the addition of D₂O in preparation for ¹H NMR analysis. Plasma samples were diluted to a final volume of 550 μl by the addition of isotonic saline solution containing 10% D₂O for the NMR field-frequency lock.

Twin samples were divided into aliquots and stored at −80° C. None of the samples underwent more than 3 freeze-thaw cycles prior to acquisition of NMR spectra. All experiments were performed under blind and randomized conditions. Plasma samples (50 μl) were made up to a final volume of 500 μl by the addition of D₂O in preparation for ¹H NMR analysis. Plasma samples were diluted to a final volume of 550 μl by the addition of isotonic saline solution containing 10% D₂O for the NMR field-frequency lock.

### ¹H NMR Spectroscopy of Plasma Samples:

Standard 1-D 600 MHz ¹H NMR spectra were acquired for all samples using a pre-saturation pulse sequence to effect suppression of the water resonance (pulse sequence: relaxation delay-90°-t₁-90°-tₘ-90°-acquire FID; Bruker Analytische GmbH, Rheinstetten, Germany). In this pulse sequence, a secondary radio frequency irradiation is applied specifically at the water resonance frequency during the relaxation delay of 2s and the mixing period (tₘ=100 ms), with t₁ fixed at 3 μs. Typically 256 transients were acquired at 300K into 32K data points, with a spectral width of 6000 Hz and an acquisition time of 1.36s per scan. Prior to Fourier transformation, the free induction decays (FID's) were multiplied by an exponential weight function corresponding to a line-broadening factor of 0.3 Hz.

### Data Reduction and Pattern Recognition Procedures:

To evaluate efficiently the metabolic variability within and between biofluids derived from patients and controls, spectra were data reduced using the software program AMIX (Analysis of MIXtures version 2.5, Bruker Rheinstetten, Germany) and exported into SIMCA-P (version-10.5, Umetrics AB, Umeå, Sweden) where a range of multivariate statistical analyses were conducted. Initially principal components analysis (PCA) was applied to the data in order to discern the presence of inherent similarities in spectral profiles. Where the classification of ¹H NMR spectra was influenced by exogenous contaminants, the spectral regions containing those signals were removed from statistical analysis. In order to confirm the biomarkers differentiating between the schizophrenia patients and matched controls, projection to latent structure discriminant analysis (PLS-DA) was employed. Where appropriate, data were subjected to one-way analysis of variance (ANOVA) using the Statistical Package for Social Scientists (SPSS/PC 13; SPSS, Chicago). Where the F ratio gave P<0.05, comparisons between individual group means were made by Dunnett T3 test at significance levels of P=0.05.

### Results

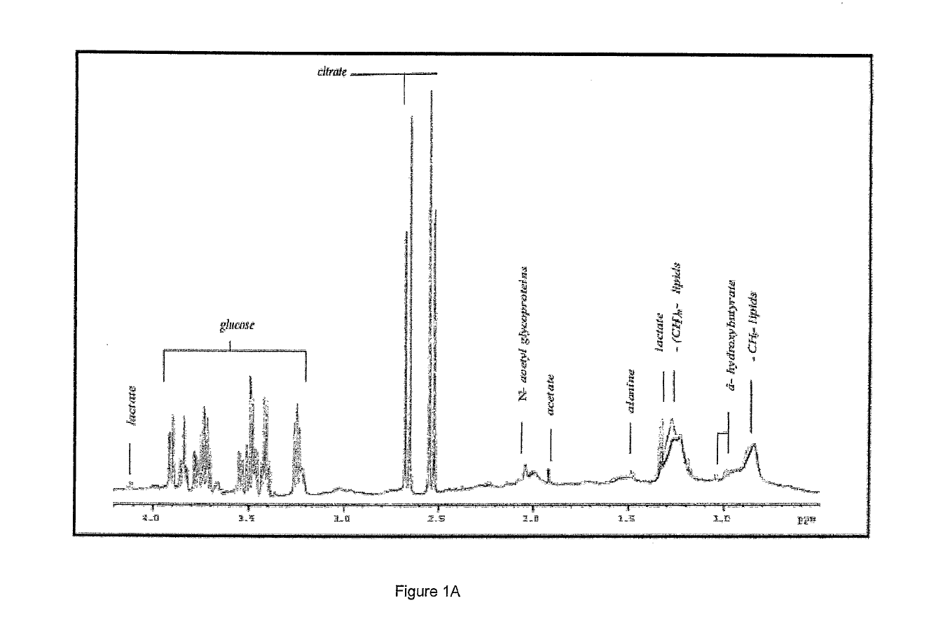

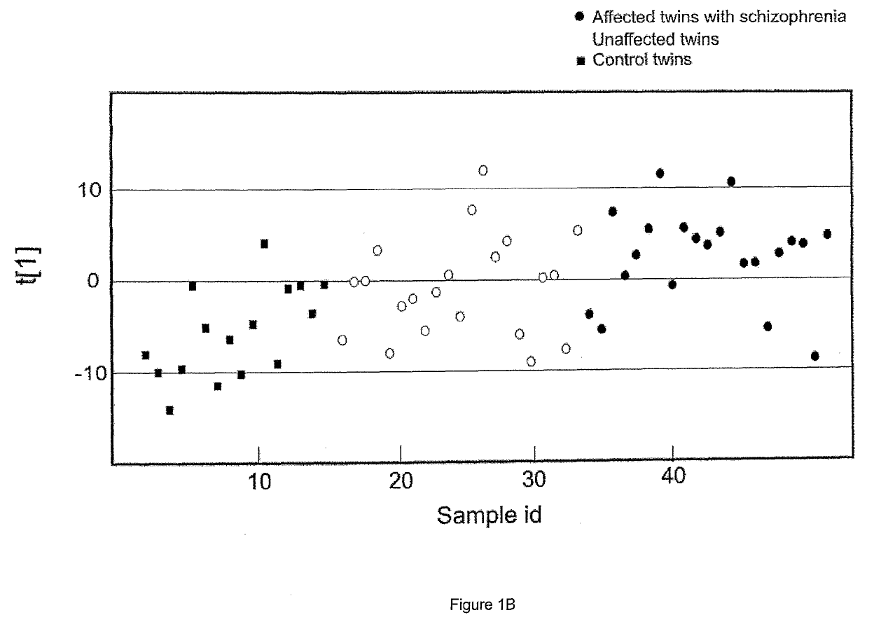

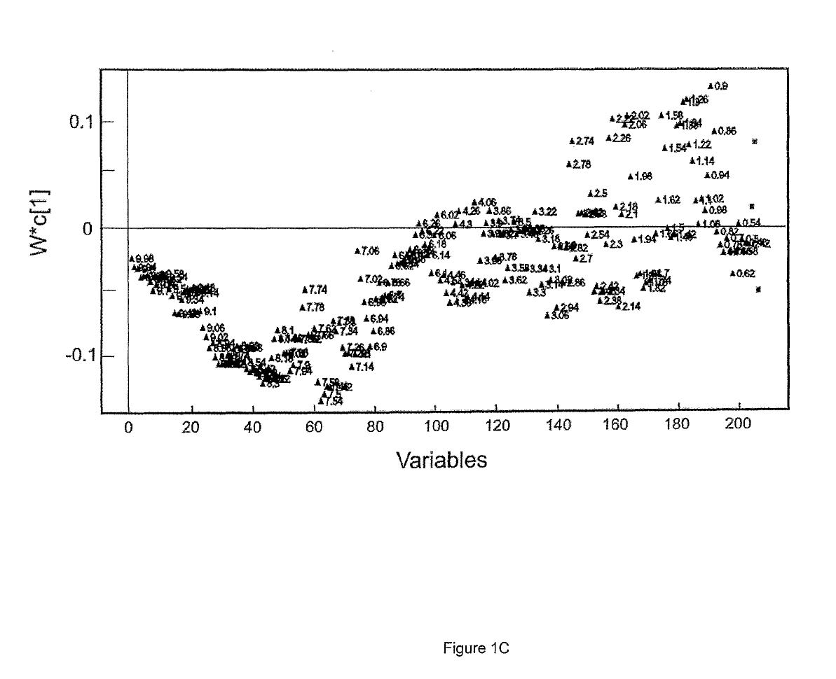

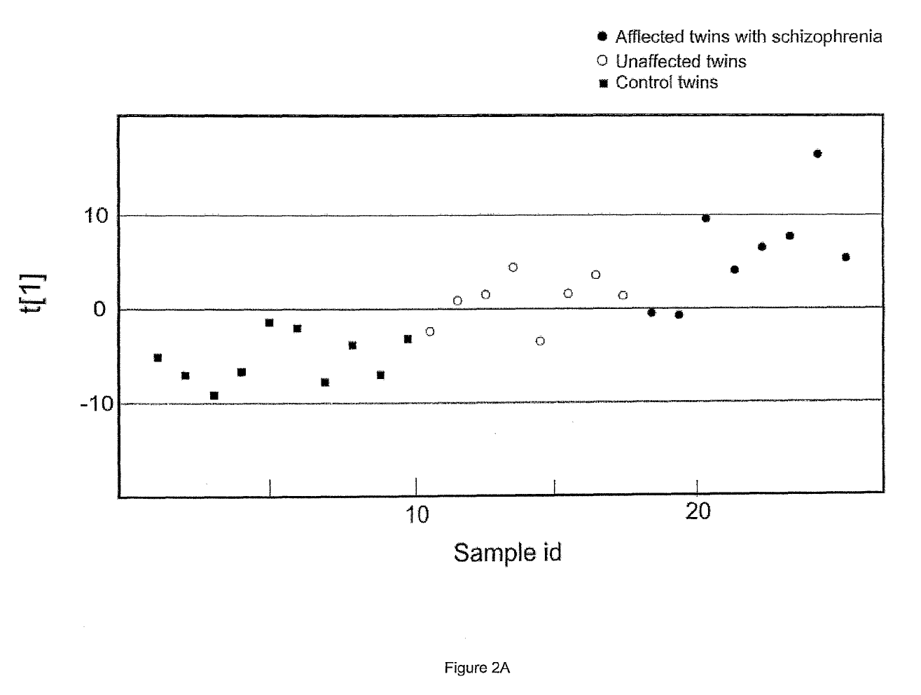

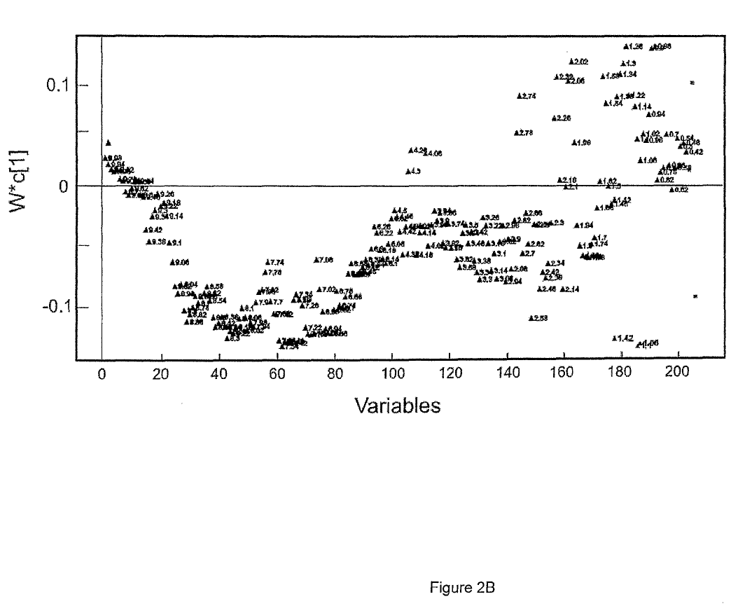

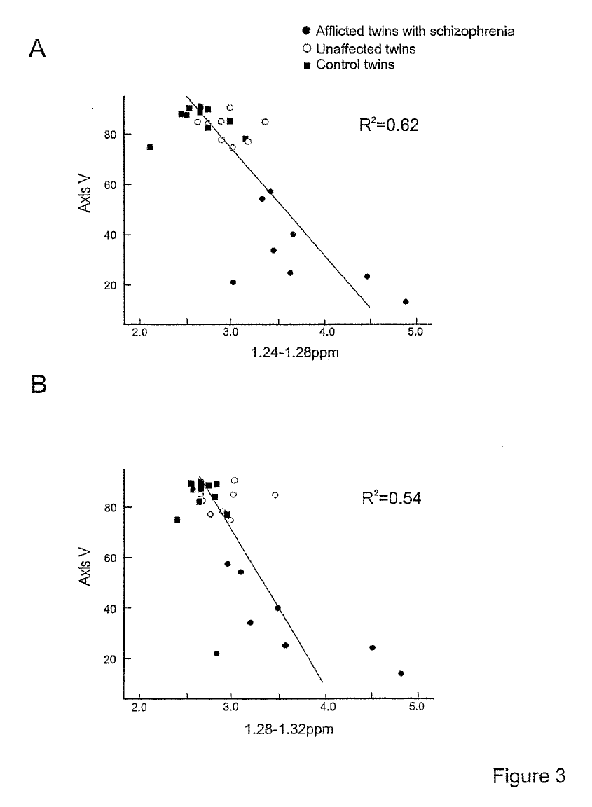

Plots of PLS-DA scores based on ¹H NMR spectra of plasma from 21 pairs of monozygotic twins discordant for schizophrenia and 16 matched control twins differentiated affected and unaffected twins from age-matched control twins (FIGS. 1A and 1B). The loading coefficients indicated that resonances from VLDL (0.92-0.88 ppm and 1.28-1.32 ppm), LDL (0.84-0.88 ppm and 1.24-28 ppm) and aromatic groups (˜δ7.5; most likely representing plasma proteins) were predominantly responsible for the separation (Table 3; FIG. 1C). Co-twins with schizophrenia showed a 23% (p=0.015; ANOVA) increase in plasma VLDL signals (1.28-1.32 ppm) compared to control twins. Corresponding unaffected co-twins were also found to have increased 1.28-1.32 ppm signals, however, differences were not quite significant for the unaffected group (p=0.18; ANOVA). LDL levels in the three groups showed a trend similar to that of the VLDL signals but, again, did not reach statistical significance (data not shown). In addition, discordant schizophrenia twins had lower plasma protein levels represented by aromatic signals around 7.5 ppm (14% and 8% reduction for the affected and unaffected co-twins respectively; p<0.01). No difference was observed in HDL signals (0.6-0.7 ppm) between the groups. Further analyses showed a much more pronounced differentiation of female twins (FIG. 2). The key chemical shifts that differentiated the groups are listed in Table 3. Interestingly, PLS-DA analyses between the female affected and healthy discordant twins alone showed that the same scores and loading plots that significantly separated the discordant twins from control twins is responsible for the separation between the discordant twins themselves. This implies that the identified metabolic alterations are a genuine disease-related signature. Furthermore, signals between 1.24-1.28 ppm (mainly LDL) correlated strongly with scores obtained from the DSM IV Axis V Global Assessment of Functioning (GAF) Scale (R²=0.62, FIG. 3), which represents one of the most widely used methods for assessing impairment among patients with psychiatric disorders. The rating is made on a scale from 1 to 100 with ratings of 1-10 representing severe impairment and ratings of 90 or more indicating superior functioning (DSMIV; Moos et al., 2002). Plasma VLDL signals (1.28-1.32 ppm) of female twins also show a strong correlation with GAF scores (R²=0.54; FIG. 3). No correlation was found when all twins or male twins alone were considered (data not shown). Age did not appear to have an effect on disease-related chemical shifts. However, antipsychotics drug exposure (measured as fluphenezine equivalent) also correlated with GAF scores and metabolic signature respectively of the female twins.

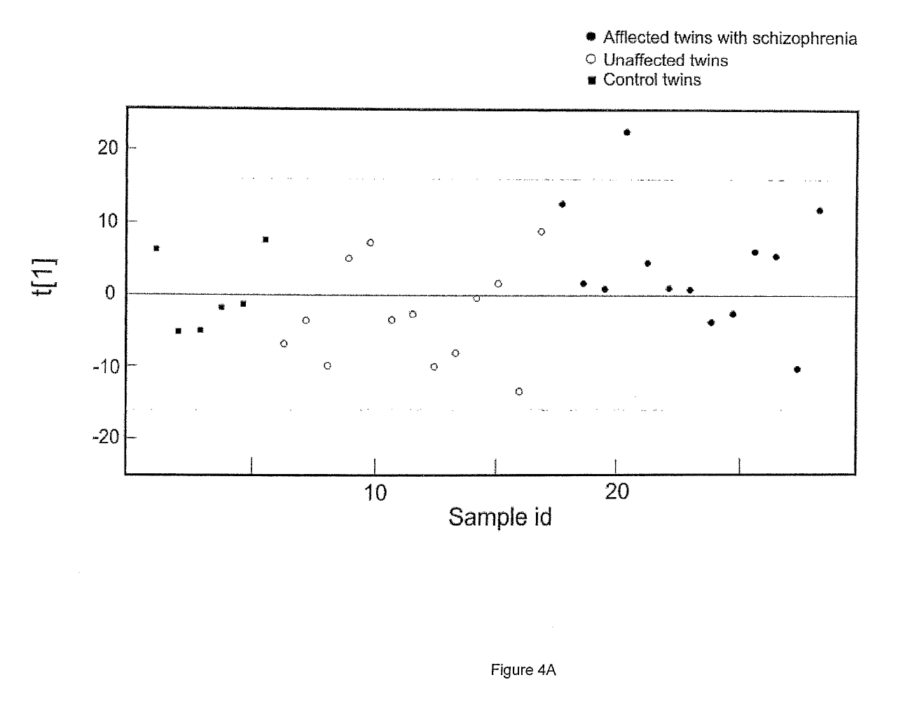

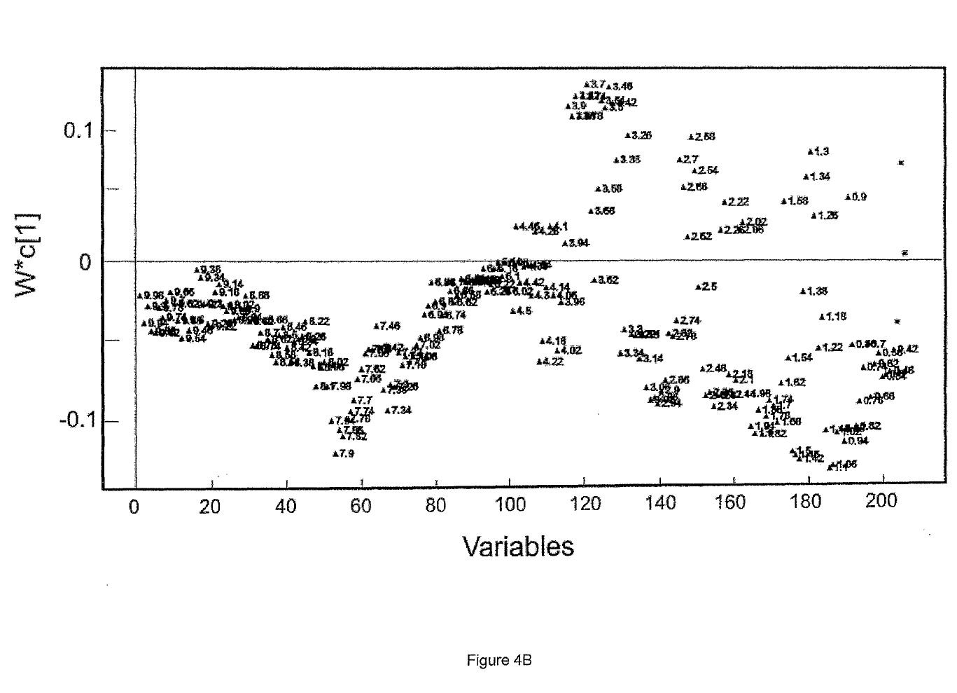

On the other hand, corresponding plots of PLS-DA scores of plasma ¹H NMR spectra derived from male twins discordant for schizophrenia showed a less prominent differentiation between affected and unaffected twins (FIG. 4A). Unlike the female twins, the loading coefficients indicated that resonances from the aromatic region, corresponding to plasma proteins, are mainly responsible for the separation amongst male twins (FIG. 4B). No correlation was found between the glucose signals and antipsychotic treatment, age, duration of illness, substance abuse and GAF scores (data not shown) for male twins. No significant difference was found between male control twins and unaffected co-twins (FIG. 4A).

### Discussion of Example 1

The present study examined the metabolic plasma profiles of a total of 42 monozygotic twins discordant for schizophrenia and 16 matched control twins using ¹H NMR in order to explore the role of genetic and environmental factors contributing to schizophrenia. The result show that signals from VLDL, LDL and aromatic regions are the most important factors differentiating ill and healthy co-twins discordant for schizophrenia from control twins. Interestingly, this differentiation was much more pronounced for female twins.

Overall, similar metabolic changes were observed in male and female schizophrenia twins, in the female group a potential predisposing disease signature was found in unaffected co-twins. This could imply a greater genetic loading for female twins. A marked sex difference in schizophrenia is a well established fact; female schizophrenia patients have, on average, a later age of onset and better prognosis. This has been attributed to protective effect of oestrogens. Women suffering from acute psychotic episodes have been shown to exhibit lower levels of oestrogen (Huber et al., 2005). Oestrogens are known to have neuroprotective properties and may reduce cell death associated with excitotoxicity as well as oxidative stress.

In female twins suffering from schizophrenia, alterations were highly associated with disease severity as well as exposure to typical antipsychotics, making it difficult to evaluate the contribution of environmental factors and drug effects. However, several lines of evidence suggest that the effect is not a drug effect: in that similar changes were identified in unaffected co-twins; also, anti-psychotic medication was not found to correlate with Global Functioning Scores in affected male twins.

One of the most interesting findings in this study is the close association of VLDL/LDL signals and Global Functioning Scores (DSMIV, Axis V) in female subjects. This is apparently the first report showing a strong correlation between a subjectively-derived clinical rating score and an objective biomarker; Thus these biomarkers are useful as an aid in diagnosis and in establishing clinical response.

TABLE 2
Demographic details of monozygotic twins

| | | | | | |
|---|---|---|---|---|---|
| | | Drug | Duration of | DSM IV | Gender |
| Total | Age | treatment^{#} | illness (yrs) | (Axis V) | (m/f) |

Twins discordant for schizophrenia

| | | | | | | |
|---|---|---|---|---|---|---|
| Affected | 21 | 33.0 ± 6.1 | 26757 ± 27320 | 12.4 ± 7.0 | 40.1 ± 13.7^{&} | 13/8 |
| Unaffected | 21 | 33.0 ± 6.1 | 0 | 0 | 82.5 ± 5.0^{&&} | 13/8 |
| Control twins | 16 | 32.1 ± 7.5 | 0 | 0 | 86.8 ± 4.5 | 6/10 |

MALE
Twins discordant for schizophrenia

| | | | | | |
|---|---|---|---|---|---|
| Affected | 13 | 32.5 ± 6.2 | 27430 ± 32607 | 13.4 ± 6.9 | 43.4 ± 11.9^{&} |
| Unaffected | 13 | 32.5 ± 6.2 | 0 | 0 | 82.1 ± 4.8^{&&} |
| Control twins | 6 | 38.7 ± 6.7* | 0 | 0 | 88.7 ± 1.5 |

FEMALE
Twins discordant for schizophrenia

| | | | | | |
|---|---|---|---|---|---|
| Affected | 8 | 33.9 ± 6.4 | 25662 ± 17537 | 11.8 ± 7.3 | 34.8 ± 15.5^{&} |
| Unaffected | 8 | 33.9 ± 6.4 | 0 | 0 | 83.3 ± 5.4 |
| Control twins | 10 | 29.3 ± 6.4 | 0 | 0 | 85.6 ± 5.4 |

*p = 0.04, control twins vs. discordant twins with schizophrenia, Oneway ANOVA
^{#}Fluphenezine equivalent.
^{&}p < 0.01, vs. the unaffected and control twins, oneway ANOVA.
^{&&}p < 0.05, vs. control twins, Oneway ANOVA.

TABLE 3
Statistical analysis of major chemical shifts that
are changed in plasma from female monozygotic twins

| Chemical shift | | Assignment^{&} | Affected twins | Unaffected twins | Control twins |
|---|---|---|---|---|---|
| 0.84-0.88 | ppm | Lipid (LDL mainly) | 2.62 ± 0.12* | 2.37 ± 0.12 | 2.25 ± 0.18 |
| 0.88-0.92 | ppm | Lipid (VLDL mainly) | 1.91 ± 0.16* | 1.72 ± 0.10 | 1.61 ± 0.10 |
| 1.24-1.28 | ppm | Lipid (LDL mainly) | 3.72 ± 0.62* | 2.96 ± 0.23^{#} | 2.64 ± 0.28 |
| 1.28-1.32 | ppm | Lipid (VLDL mainly) | 3.15 ± 0.98* | 2.31 ± 0.36 | 1.97 ± 0.19 |
| ~7.5 | ppm | Aromatic groups | 0.142 ± 0.009* | 0.153 ± 0.007^{#} | 0.166 ± 0.008 |

Data are shown as mean ± S.D.
^{&}The assignments of signals are based on a study by Nicholson and Foxall¹⁴.
*p < 0.05 vs. unaffected twins and control twins; Oneway ANOVA
^{#}p < 0.05 vs. control twins; Oneway ANOVA

### Example 2

Extensive protein/peptide profiling analysis of CSF samples from a total of 139 CSF samples (80 controls and 59 first onset, drug-naïve schizophrenia patients) was performed using SELDI mass spectrometry in combination with computerized pattern recognition analysis. Highly significant and reproducible differences were found in samples obtained from first-onset, drug-naïve patients with a diagnosis of paranoid schizophrenia as compared to age-matched controls.

TABLE 4
Demographic details of subjects in
the first CSF SELDI experiment

| | | Gender |
|---|---|---|
| | Age* | (male/female) |
| First-onset, drug-naïve | 28.7 ± 9.2 | 30/11 |
| schizophrenia patients | | |
| Healthy volunteers | 28.3 ± 7.0 | 24/16 |

*Data are shown as average ± S.D.

TABLE 5
Demographic details of subjects in the CSF validation sample set

| | | Gender |
|---|---|---|
| | Age* | (male/female) |
| First-onset, drug-naïve | 27.6 ± 7.9 | 10/8 |
| schizophrenia patients | | |
| Healthy volunteers | 27.3 ± 3.8 | 20/20 |

*Data are shown as average ± S.D.

TABLE 6
Demographic details of subjects in the analysis
in FIG. 3C (Western blot analysis of post-mortem
analysis for transthyretin expression).

| | | Gender | Fluphenazine mg. |
|---|---|---|---|
| | Age* | (male/female) | Equivalents |
| Control subjects | 46.2 ± 6.0 | 4/1 | N/A |
| Schizophrenia patients | 44.8 ± 8.8 | 3/2 | 70,000 ± 70,200 |

*Data are shown as average ± S.D.

TABLE 7
Sensitivity and specificity of PLS models calculated
from the two independent experiments

| | | (Healthy volunteers/schizophrenia) | |
|---|---|---|---|
| | Samples | Sensitivity¹ | Specificity² |
| Initial experiment | (40/41) | 80% | 95% |
| Validation experiment | (40/18) | 90% | 98% |

¹Sensitivity is defined as the proportion of true positives it detects of all the positives.
²Specificity is defined as the proportion of true negatives it detects of all the negatives.

### Clinical Samples

The Ethical committee of the Medical Faculty of the University of Cologne reviewed and approved the protocol of this study and the procedures for sample collection and analysis. All study participants gave their written informed consent. All clinical investigations were conducted according to the principles expressed in the Declaration of Helsinki. CSF and serum samples were collected from drug-naïve patients diagnosed with first episode paranoid schizophrenia or brief psychotic disorder due to duration of illness (DSM-IV 295.30 or 298.8, n=59) and from demographically matched healthy volunteers (n=80) (Tables 4 and 5). Fresh-frozen prefrontal cortex tissue (Brodmann area 9) from gray matter of 8 schizophrenia and 8 well-matched control individuals was obtained from the Neuropathology Consortium of the Stanley brain collection (Stanley Medical Research Institute, USA).

### Preparation of CSF Samples for SELDI Analysis

5 μl of each CSF sample was applied to the chips with different chemical properties at various pH conditions. The best condition was chosen at pH 9.0 on strong anion exchanger Q10 chip based on number and separation of peaks resolved. Briefly, the array spots were pre-activated twice with binding buffer (100 mM Tris-HCl, pH9.0) at room temperature for 10 min on a shaker (frequency=600 rpm). 50 μl binding buffer composition was added into each protein spot prior to the addition of 5 μl CSF sample. The protein chips were incubated on a shaker for 60 min at room temperature. The chips were washed twice with binding buffer and once with H₂O, and then air-dried. The chips were then sequentially treated twice with 0.6 μl of a 100% saturated sinapinic acid (3,5-dimethoxy-4-hydroxycinnamic acid) in 50% acetonitrile and 0.5% trifluoroacetic acid. The chips were analyzed with the Ciphergen ProteinChip Reader (Ciphergen ProteinChip System Series 4000). Each sample was analyzed twice to confirm reproducibility in identifying the differentially expressed proteins.

### SELDI-TOF-MS Analysis

The arrays were analyzed with the Ciphergen ProteinChip System Series 4000 (Ciphergen Biosystems, USA). Mass spectra of proteins were generated by using an average of 254 laser shots at a laser intensity of 1800 arbitrary units. For data acquisition, the detection size range was between 3 and 200 kDa. The laser was focused at 10 kDa. The mass-to-charge ratio (m/z) of each of the proteins captured on the array surface was determined according to externally calibrated standards (Ciphergen Biosystems; USA): bovine insulin (5,733.6 Da), human ubiquitin (8,564.8 Da), bovine cytochrome c(12,230.9 Da), bovine superoxide dismutase (15,591.4 Da), horseradish peroxidase (43,240 Da) and BSA (66,410 Da). The data were analyzed with PROTEINCHIP data analysis software version 3.0 and Ciphergen Express Software 3.0 (Ciphergen Biosystems; USA). The Ciphergen Express Software 3.0 was used to compile all spectra and autodetect quantified mass peaks. Peak labelling was completed by using second-pass peak selection with 0.2% of the mass window, and estimated peaks were added. The peak information of all spectra was exported for further statistic analyses.

### Peptide and Protein Identification

Identification of the schizophrenia specific peptides was performed by a combination of purification step (either on-chip) followed by C18 Zip-Tip purification. Typically, 10 μl CSF samples from each the control and schizophrenia groups were subjected to Q10 protein chips at pH 9.0 (50 mM Tris-HCl). Proteins/peptides bound to the chip were eluted with 5 μl elution buffer (30% acetonitrile, 50 mM sodium acetate pH3.0) by pipetting and was desalted using a C18 Ziptip according to manufacturer's manual. The peptides eluted with 0.1% formic acid/50% aqueous acetonitrile (2 μl) were further examined by MALDI mass spectrometry for confirmation of the peak in CSF samples from schizophrenia patients. The eluted peptides were also loaded into a C18 nano-column linked with ESI-MS/MS (Applied Biosystems, USA) for de novo sequencing.

For protein biomarkers, CSF proteins were purified from pooled CSF by a combination of anion exchange chromatography (HyperD F; Ciphergen Biosystems; USA) followed by SDS-PAGE. The bands around the matched mass were cut out and the proteins from ⅓ of the excised protein band was eluted passively using previous described method²⁰ to confirm the mass in the spectrum. The rest of the protein band was in-gel digested with trypsin (1:50; Promega, UK) overnight at room temperature. The resulting peptide mixtures were then sequenced using LC-MS/MS (Applied Biosystems, USA).

S-cysteinylated or S-glutathionylated isoforms (which are isoforms are generated in vivo) of proteins were confirmed by comparing the spectra before and after on-chip reduction using β-mecaptoethanol. In brief, CSF protein and peptide binding was performed as described above and in the final step each spot was washed with 100 ul 1 mM HEPES pH 7.5. The proteins and peptides on the chips were then reduced with 1/40 β-mecaptoethanol (1 μl) for 30 min at room temperature. 1 ml of water was added onto each spot and evaporated. This procedure was repeated twice. Matrix was then added on and data were acquired using ProteinChip Reader (Ciphergen ProteinChip System Series 4000).

### Quantitative Analysis of Transthyretin in Human Serum Samples by Enzyme-Linked Immunosorbent Assays (ELISA)

Samples were defrosted from −80° C. and vortexed for 10 min before experimental work. All samples were assayed blind to the clinical conditions. The identity of all subjects was blind by a code number until all biochemical analyses were completed.

Transthyretin standard (Sigma, UK), controls and patient-derived human serum samples were diluted 1000 times with phosphate buffered saline, pH 7.4, (Sigma, UK), Transthyretin standard and samples were then loaded onto ELISA Maxisorb plates (Nunc™, Denmark) and incubated for 1 h. All samples were tested in duplicate. After washing with Washing buffer (0.03% Tween 20 in PBS), the plates were blocked with 5% skimmed milk powder for 60 min. 100 μl transthyretin antibody (DakoCytomation, Denmark, 1:500 diluted in 2.5% skimmed milk powder) was incubated in 96-well plates for 60 min. The plates were washed four times with Washing buffer followed by addition of 100 μl secondary antibody (anti-rabbit HPP-linked IgG (Cell Signalling, UK; 1:2000) to each well and incubated for 60 min. After washing with Washing Buffer three times, 100 μl substrate (TMB One solution, Promega) was added into each well and the mixture was incubated at room temperature for 10 min. The plate was read at 450 nm (BIO-RAD, Model 680).

### Western Blot Analysis

The preparation of human brain samples for Western blot analysis and the details of performing Western blotting were as described previously²¹. In brief, equivalent amounts of protein (30 μg per sample) were resolved electrophoretically on 10% polyacrylamide gels and transferred onto nitrocellulose, which was then incubated with primary antibody (anti-transthyretin, DakoCytomation) in 3% milk-PBS overnight at 4° C., followed by incubation of a secondary antibody (HRP conjugated anti-rabbit secondary antibody (Cell Signaling, 1:2500) at room temperature for 1 hr. Enhanced chemiluminescence (LumiGlu™, Cell Signaling) was used to detect signals from the blot. Consistency of protein loading and transfer was determined by Ponseau S staining.

### Statistic Analysis

Multivariate statistical analysis including principal component analysis (PCA), partial least squares discriminate analysis (PLS-DA) and PLS were employed to summarize the data output from Ciphergen Express. Holdout cross-validation was performed three times so that the sensitivity and specificity of the PLS model could be estimated. In each of the three rounds of holdout cross-validation, one third of the samples were randomly selected to form the validation data and the remaining samples were used as the training data. All multivariate analyses were performed using SIMCA-P+ 10 (Umetrics AB, Sweden). Sensitivity is defined as the proportion of true positives it detects of all the positives and specificity is defined as the proportion of true negatives it detects of all of the negatives. Where appropriate, t test was performed using the Statistical Package for Social Scientists (SPSS/PC+; SPSS, Chicago).

Alterations of CSF Protein/Peptide Profiles in First-Onset, Drug-Naïve, Paranoid Schizophrenia Patients

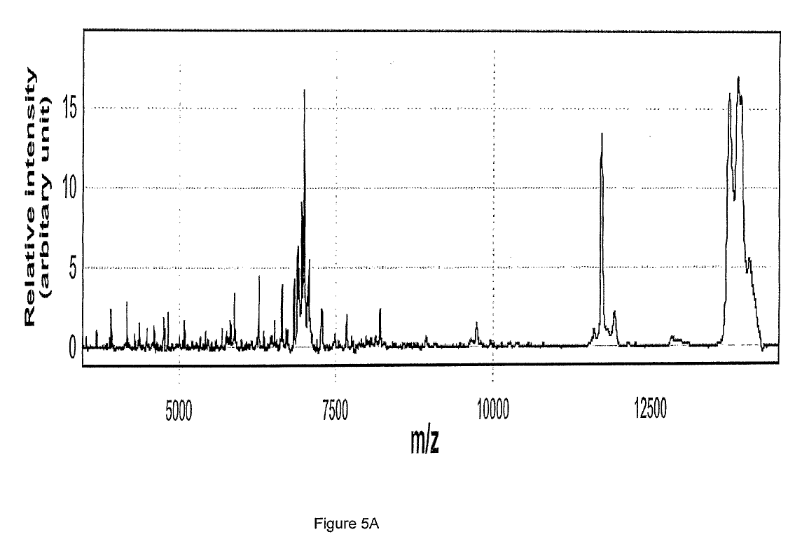

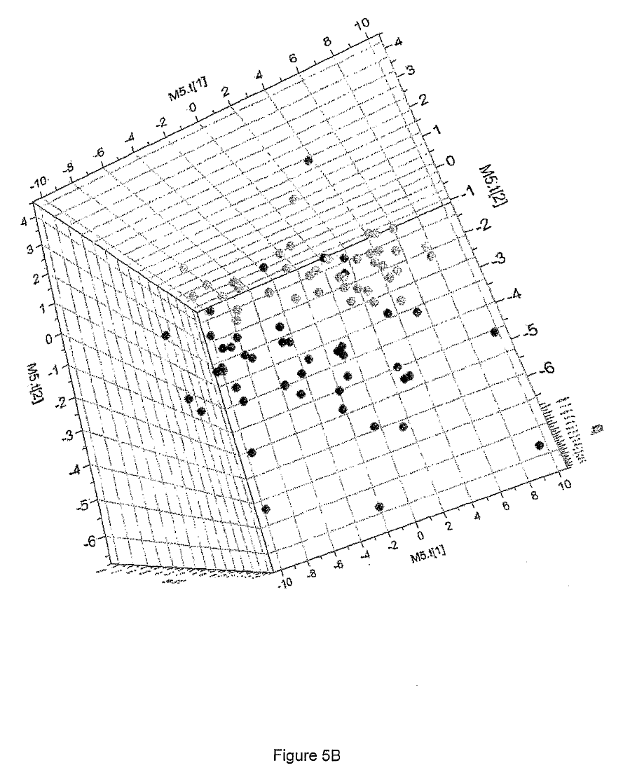

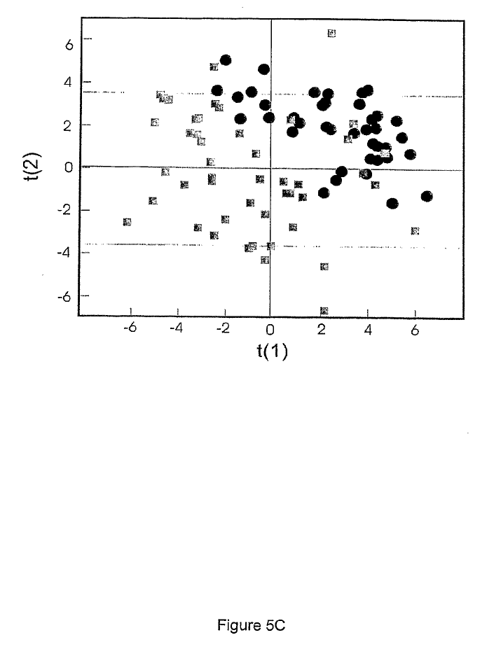

In a first set of experiments protein/peptide profiles of CSF samples from 41 first-onset, drug-naïve, paranoid schizophrenia patients and 40 demographically matched healthy volunteers were examined using SELDI mass spectrometry. CSF proteins and peptides were profiled using Q10 (strong anion exchanger) chips at pH 9.0. An example of the CSF protein/peptide profile of a healthy volunteer is shown in FIG. 5A. Approximately 75 peaks can be readily detected with a signal to noise ratio >5 under this Q10 protein chip binding condition. Plots of PLS-DA scores based on SELDI spectra of CSF samples showed a clear differentiation between healthy volunteers and drug-naïve patients with first onset, paranoid schizophrenia (FIGS. 5B and C). Similar results were found using principle component analysis (data not shown). The loading plot showed significant reductions in clusters of peaks between 13,600-14,000Da. The sensitivity and specificity of this model based on holdout cross validation was 80% and 95%, respectively (Table 7).

### Identification of the 13.6-14 kDa Protein Cluster as Transthyretin

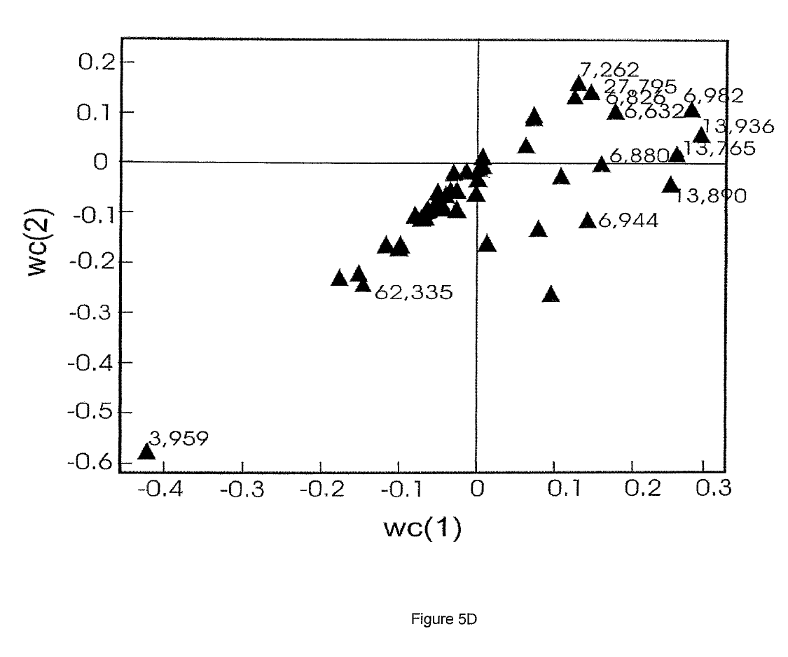

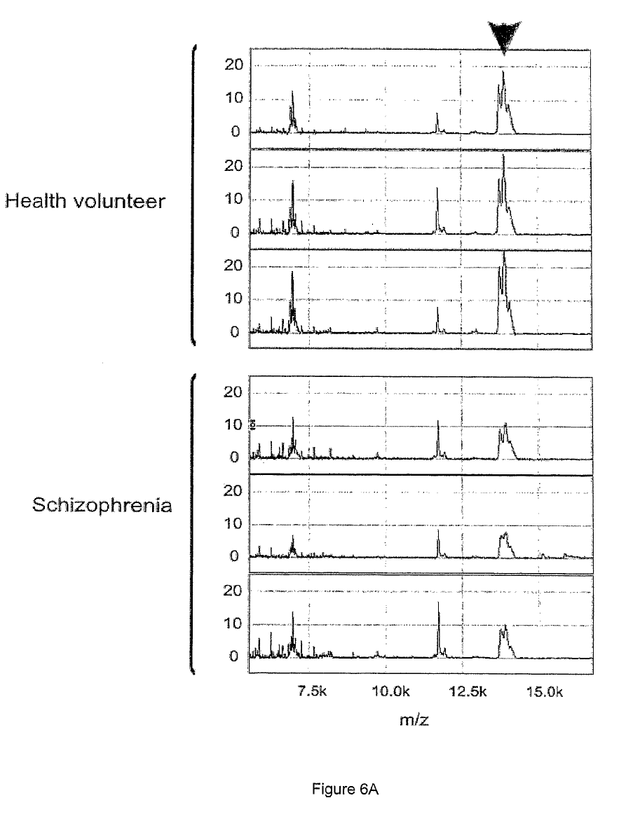

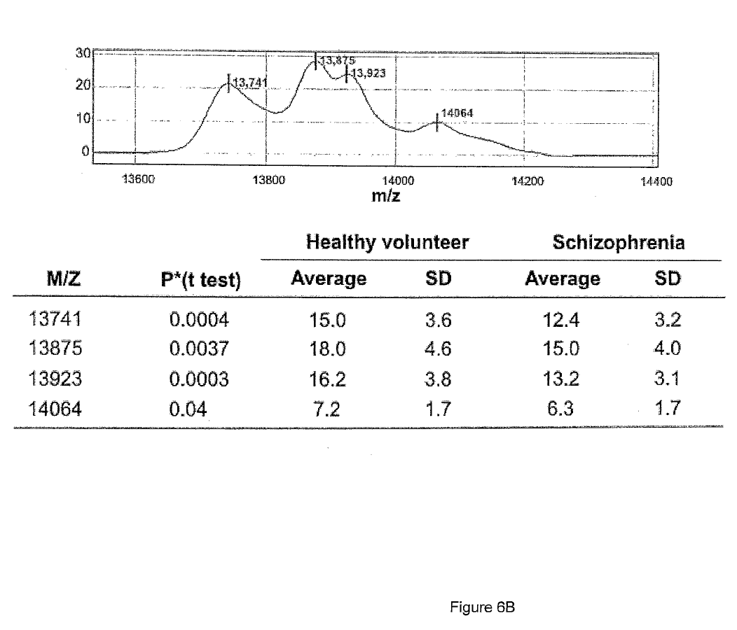

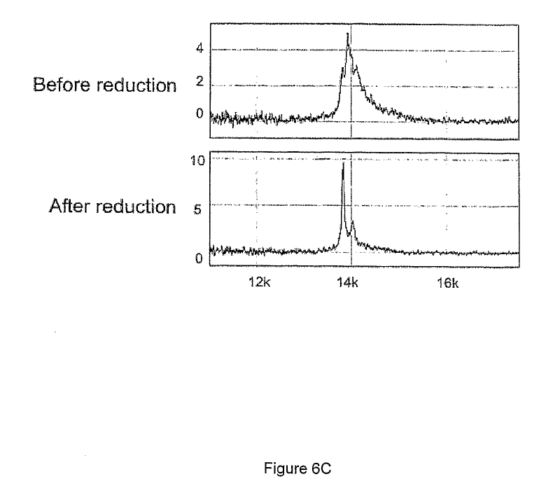

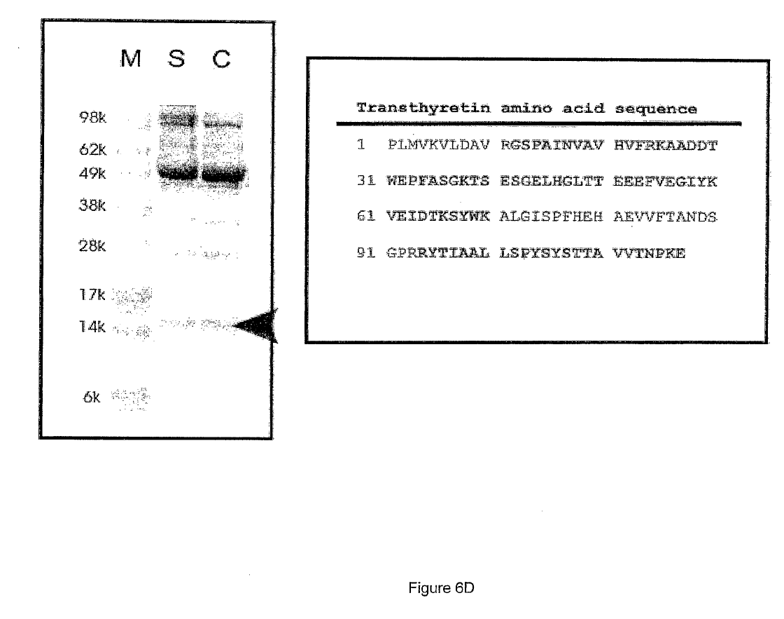

The protein cluster between 13.6-14.1 kDa contained four peaks (FIG. 6B), three of which were consistently down-regulated in CSF from first onset, drug-naïve schizophrenia patients (p<0.01; FIG. 6B, bottom panel). Studies have suggested that these peaks may be from S-cysteinylated or S-glutathionylated derivatives of transthyretin protein^{22,23}, a thyroid hormone-binding protein that transports thyroxine from the bloodstream to the brain. On-chip reduction of CSF peptide/protein performed using β-mercaptoethanol at room temperature showed that the three peaks 13,741, 13,875, and 13,923Da were reduced to a single peak (FIG. 6C), confirming they were derived from the same protein. To identify the protein, a pair of CSF samples from a healthy volunteer and a schizophrenia patient were applied to an anion exchanger column (HyperD) and eluted with pH 9-pH 3 buffers. A major band ˜13-15 kD was eluted in the pH3 fraction (FIG. 6D, left panel). The band was confirmed to be the peak cluster around 13.6-14 kDa in the SELDI spectrum by eluting the protein from the band and running on a NP20 chip to match the mass (FIG. 66E). This protein was then digested with trypsin and sequenced using LC-MS/MS. The protein was identified as transthyretin (FIG. 6D, right panel).

Down-Regulation of Transthyretin in Serum Samples from the Same Subjects as Well as Prefrontal Cortex Tissue from Schizophrenia Patients

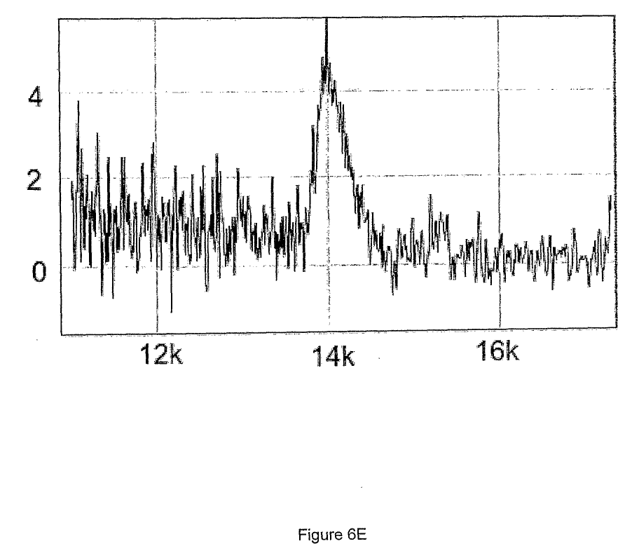

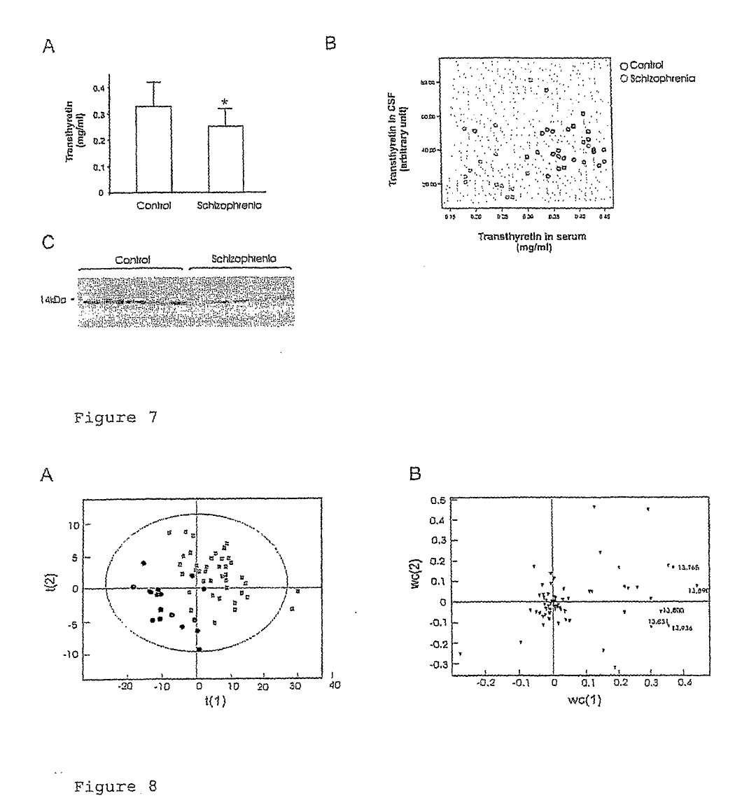

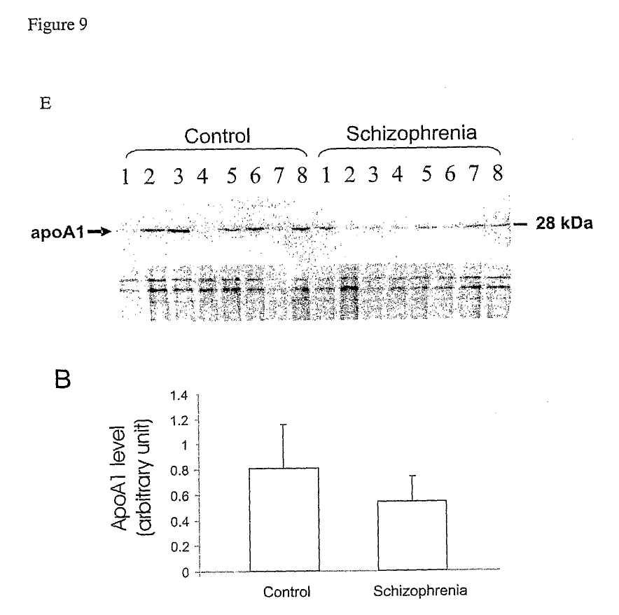

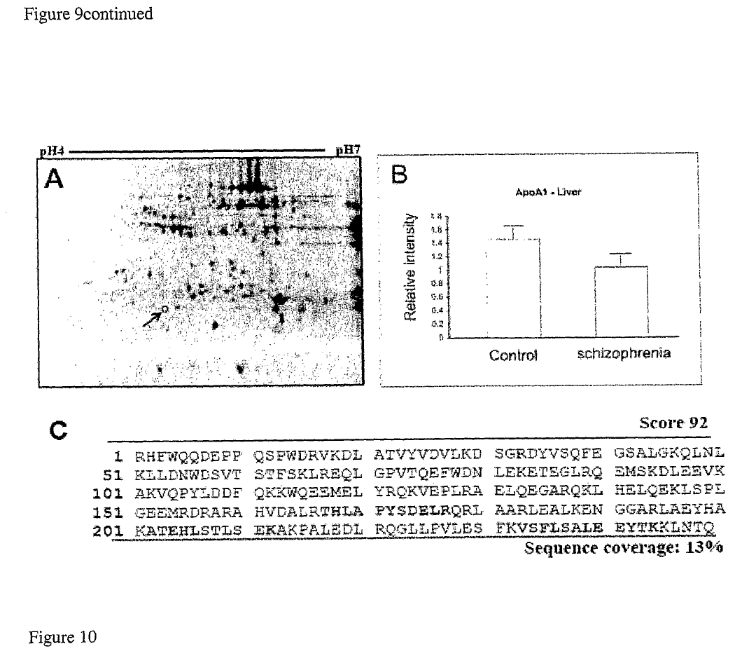

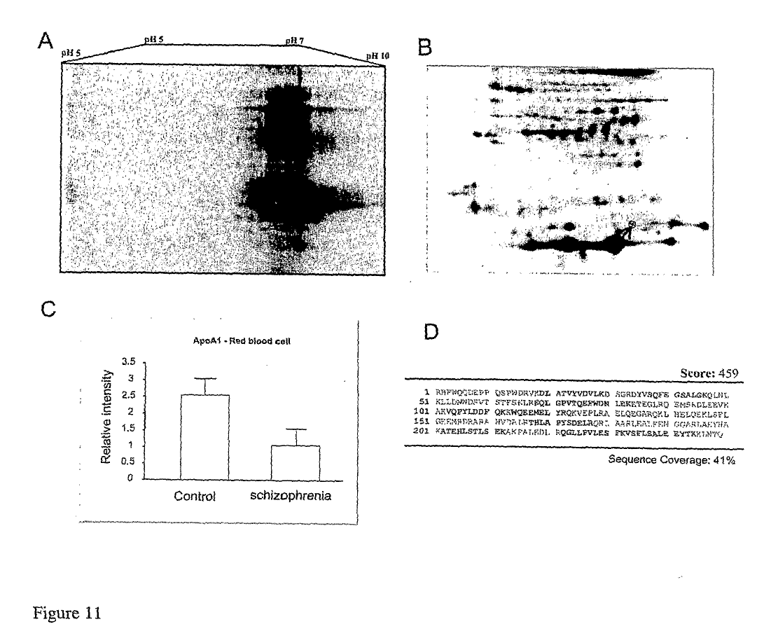

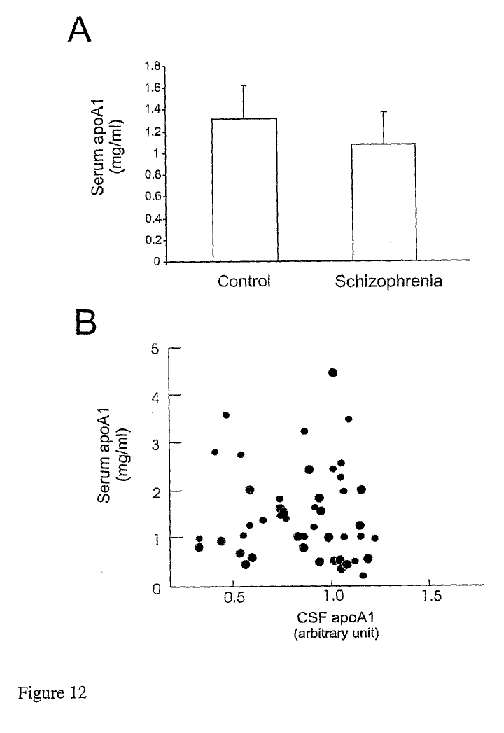

It has been estimated that 3% of transthyretin in the ventricular CSF and 10% of the transthyretin in lumbar CSF are derived from blood. To evaluate the contribution of blood transthyretin to the changes found in CSF in schizophrenia, serum transthyretin levels taken from the same individuals (at the same time when CSF was collected) who had been studied in FIGS. 5 and 8 (for demographics, see Tables 4 and 5) were investigated using ELISA. A moderate but significant decrease of transthyretin in sera was found from schizophrenia patients compared to controls (15% decrease, p=0.0007, t test) (FIG. 7A). However, no correlation between CSF and serum transthyretin levels from the same individuals was found, suggesting that transthyretin levels are regulated independently in CSF and serum (FIG. 7B). Transthyretin levels were decreased in both CSF and serum samples. However, there is no correlation of CSF transthyretin levels and serum transthyretin level.

Interestingly, a ˜40% down-regulation of transthyretin in post-mortem prefrontal cortex from schizophrenia patients as compared to controls using Western blot was found (FIG. 7C).

### Validation of Protein/Peptide Biomarkers in an Independent Sample Set

The biomarker model in FIG. 5 was validated using an independent sample set consisting of a further 18 first-onset, drug-naïve schizophrenia patients and 40 demographically matched healthy volunteers. These samples were run using identical conditions as in the previously described experiment. PLS-DA scores and loadings plots showed a very similar result as found in FIG. 5 with in the cluster of 13,600-14,000 proteins (FIG. 8). This suggests that these identified alterations in CSF proteins and peptides are a consistent finding and thus may reflect genuinely the early pathophysiology of schizophrenia. The sensitivity and specificity of this model was 95% and 98%, respectively (Table 7).

### Discussion of Example 2

Initial analysis of SELDI spectra of a total of 81 CSF samples (41 schizophrenia; 40 controls) showed a differential distribution of samples from drug-naïve patients with first onset paranoid schizophrenia away from healthy volunteer samples (FIGS. 5B, 5C and 5D). The protein/peptide profile of CSF was found to be characteristically altered in paranoid schizophrenia patients and a key alteration was the down-regulation of transthyretin around 14 kDa. These schizophrenia specific protein/peptide changes were replicated/validated in an independent sample set (n=58) using identical conditions (FIG. 8). Both experiments achieved an astonishingly high specificity (rate of true negative) of 95/98% and a sensitivity of 80/90%, respectively (Table 7). This means that virtually no control samples clustered with the schizophrenia group (FIGS. 5B and 5C). For a high diagnostic validity and consequent therapeutic interventions an accurate identification of those individuals who truly have the disease is most critical.

A moderate but consistent decrease of transthyretin was observed in CSF from first onset schizophrenia patients. ELISA results on the serum samples collected from the identical individuals whose CSF samples were investigated in this study showed that there is a ˜15% decrease in transthyretin levels in serum (p=0.0007, t test; FIG. 7A), however, there was no correlation between the levels of serum transthyretin and SELDI signals from CSF transthyretin, suggesting that liver derived transthyretin may not contribute to the down-regulation in CSF (FIG. 7B). Experiments perfusing isolated sheep brains showed that all newly synthesized transthyretin was secreted from the choroid plexus towards the ventricles. The synthesis of this protein is required for the transport of thyroxine¹⁶. Thus, the decreased level of transthyretin in CSF suggests a lowered thyroxine transport in brains of schizophrenia patients. Indeed, the results found in this study showing a down-regulation in transthyretin protein in post-mortem brain tissue from schizophrenia patients (FIG. 7C) further support this notion. It is noteworthy that thyroid dysfunction is relatively common in patients with schizophrenia^{24,25} and indeed other psychiatric disorders²⁶, possibly genetically linked to the disorders. In addition, in patients with severe forms of both hypo- and hyper-thyroidism, psychotic symptoms may occur and the clinical picture frequently resembles that of schizophrenia²⁷, which may imply that an increase in CNS thyroxine function may be linked. Interestingly, long-term administration of clozapine has been shown to induce transthyretin expression in rat hippocampus and cerebral cortex¹⁸, implying that clozapine enhances CNS thyroxine function in light of the results herein, supporting the clinical relevance of transthyretin in the early pathophysiology of schizophrenia.

The application of SELDI mass spectrometry can provide an efficient means for early diagnosis of paranoid schizophrenia.

### Example 3

### Clinical Samples

The protocols of this study including procedures for sample collection and analysis were approved by ethical committees. Informed consent was given in writing by all participants and clinical investigations were conducted according to the principles expressed in the Declaration of Helsinki. CSF and serum samples were collected from drug-naïve patients diagnosed with first episode paranoid schizophrenia or brief psychotic disorder due to duration of illness (DSM-IV 295.30 or 298.8; n=41 for CSF; n=35 for serum; Table 8) and from demographically matched healthy volunteers (n=40 for CSF; n=63 for serum; Table 8).

For post-mortem studies, fresh-frozen prefrontal cortex tissue (Brodmann area 9; 8 schizophrenia and 8 well matched control individuals) and liver samples (15 schizophrenia and 15 well matched controls) were obtained. The demographic details are listed in Table 8.

For red blood cell (RBC) experiments, a total of 40 blood samples (7 first-onset, drug-naïve schizophrenia patients and 13 schizophrenia patients treated with atypical antipsychotic medication as well as 20 demographically-matched healthy volunteers, see Table 8 for demographic details) were collected from two centres using an identical sample collection procedure.

TABLE 8
Demographic details of schizophrenia and control subjects

| | | Schizophrenia | Control |
|---|---|---|---|
| CSF | Sample size | n = 41 | n = 40 |
| | Age | 28.7 ± 9.2 | 28.3 ± 7.0 |
| | Gender (M/F) | 30/11 | 24/16 |
| Liver | Sample size | n = 15 | n = 15 |
| | Age | 44.7 ± 6.2 | 42.5 ± 6.9 |
| | Gender (M/F) | 9/6 | 9/6 |
| | Fluphenazine mg Equivalents | 93,460 ± 88,322 | N/A |
| RBC | Sample size | n = 20 | n = 20 |
| | Age | 34.5 ± 9.2 | 38.8 ± 11.0 |
| | Gender (M/F) | 17/3 | 15/5 |
| Serum | Sample size | n = 35 | n = 63 |
| | Age | 28.0 ± 8.8 | 27.6 ± 5.7 |
| | Gender (M/F) | 21/14 | 33/30 |
| Brain | Sample size | n = 8 | n = 8 |
| | Age | 43.0 ± 6.5 | 47.8 ± 7.1 |
| | Gender (M/F) | 5/3 | 6/2 |
| | Fluphenazine mg Equivalents | 95,575 ± 97,069 | N/A |

### Preparation of RBC Samples

Blood samples were collected in anticoagulant EDTA tubes prior to cell isolation and protein extraction (see below). To purify RBCs, 40 ml of freshly drawn blood was diluted with 40 ml of PBS. The diluted blood was gently layered on half volume of a density gradient separation medium (HISTOPAQUE®-1077, Sigma) and centrifuged at 750×g for 10 min. Isolated RBC were then collected from the bottom of the tube and frozen at −80° C. RBC were lysed with erythrocyte lysis buffer (Qiagen, UK) in 1:5 ratios at 4° C. for 15 minutes. Proteins were extracted by precipitation using 100 mM ammonium acetate in methanol overnight at −20° C. and resuspended in ASB14 buffer (8 M urea, 2% ASB14, 5 mM magnesium acetate, 20 mM Tris-HCl, 1% Triton-X100, pH 8) containing complete protease inhibitor cocktail (Roche, Switzerland) and phosphatase inhibitors (1 mM sodium pyrophosphate, 1 mM sodium orthovanadate, 10 mM β-glycerophosphate, and 50 mM sodium fluoride). Protein concentration was determined using a detergent-compatible protein assay kit (BioRes). The highly abundant protein, haemoglobin, was first pre-fractionated from the RBC proteome using a ZOOM® IEF Fractionator (Invitrogen). This is a simple and convenient method to reproducibly fractionate cell lysate on the basis of isoelectric point (pI) using solution phase isoelectric focussing (IEF). Fractionated proteins with a pI between 6.2 and 10, containing Hb were discarded. The remaining fractions from each individual patient/control (pI 3-6.2) were pooled and proteins were re-extracted by ammonium acetate precipitation and subjected to 2D-DIGE analysis.

### 2D-DIGE Analysis

2D-DIGE analyses of liver samples were performed using 24 cm, pH4-7, IDG DryStrips. The detail procedures are as described previously (18).

### CSF Protein Profiling Using SELDI-TOF Analysis and Protein Biomarker Identification

5 μl samples of each CSF was applied to protein chips with different chemical properties at various pH conditions. The best condition was chosen at pH 9.0 on strong anion exchanger Q10 chip, based on number and separation of peaks resolved. Briefly, the array spots were pre-activated twice with binding buffer (100 mM Tris-HCl, pH 9.0) at room temperature for 10 minutes on a shaker (frequency=600 rpm). 50 μl binding buffer was added into each spot prior to the addition of the 5 μl CSF sample. The protein chips were incubated on a shaker for 60 min at room temperature, then washed twice with binding buffer, once with H₂O, and air-dried. The chips were then sequentially treated twice with 0.6 μl of a 100% saturated sinapinic acid (3,5-dimethoxy-4-hydroxycinnamic acid) in 50% acetonitrile and 0.5% trifluoroacetic acid. The chips were analyzed using the Ciphergen ProteinChip Reader (Ciphergen ProteinChip System Series 4000). Each sample was analyzed twice to confirm reproducibility in identifying the differentially expressed proteins. Mass spectra of proteins/peptides were generated by using an average of 254 laser shots at a laser intensity of 1800 arbitrary units. For data acquisition, the detection size range was between 3 and 200 kDa. The laser was focused at 10 kDa. The mass-to-charge ratio (m/z) of each of the proteins captured on the array surface was determined relative to external calibration standards (Ciphergen Biosystems; USA): bovine insulin (5,733.6 Da), human ubiquitin (8,564.8 Da), bovine cytochrome c (12,230.9 Da), bovine superoxide dismutase (15,591.4 Da), horseradish peroxidase (43,240 Da) and BSA (66,410 Da). The data were analyzed with PROTEINCHIP data analysis software version 3.0 and using Ciphergen Express Software 3.0 (Ciphergen Biosystems; USA). The Ciphergen Express Software 3.0 was used to compile all spectra and autodetect quantified mass peaks. Peak labelling was completed by using second-pass peak selection with 0.2% of the mass window, and estimated peaks-were added. The statistic analyses of peak information were performed using Ciphergen Express Software 3.0.

For identification of protein biomarkers, CSF proteins were purified from pooled CSF by a combination of anion exchange chromatography (HyperD F; Ciphergen Biosystems; USA) followed by SDS-PAGE. The band expected correspond to the SELDI peak was cut from the gel and the gel band was in-gel digested with trypsin (1:50; Promega, UK) overnight at room temperature. The resulting peptide mixtures were then analyzed by LC-ESI-MS/MS (QSTAR, Applied Biosystems, USA), and the protein identified by database searching using Mascot software (Matrix Science, London). To confirm the gel band is the protein of interest, an antibody capture experiment was performed. Briefly, 2 μl of antibody (0.2 mg/ml) was coupled to RS100 reactive chip surface, followed by blocking with 2M Tris-HCl (pH8.0) at room temperature according to the manufacturer's protocol. 5 μl CSF samples were then applied directly to spots with or without antibody coupling and incubated for 1 hr. After washing 5 times with 10 μl HEPES buffer (50 mM, pH7.2), the chips were analyzed with Ciphergen ProteinChip System Series 4000.

### Western Blot Analysis

Western blot analysis of brain tissues has been described previously (32). Briefly, after determining the protein concentration, samples were diluted in sample buffer (Invitrogen), to a final concentration of 4 mg/ml. 30 μg of protein was loaded into each well and separated on 4%-12% SDS pre-cast-gel (Invitrogen) alongside ApoA1 standard (Sigma) as a positive control. Separated proteins were transferred onto nitrocellulose membranes at room temperature. The nitrocellulose membranes were incubated with blocking solution (5% dried skimmed milk) for 60 minutes at room temperature followed by incubation with anti-human ApoA1 polyclonal antibody (1:1000) (CalBiochem) overnight at 4° C. Membranes were washed four times with wash buffer and then incubated with horseradish peroxidase-conjugated secondary antibody (Cell Signalling, 1:5000) at room temperature for 1 hr. Chemiluminescent visualization (GE Heathcare) was used to visualize the signals.

### ELISA

Serum samples were randomized and the identity of all subjects was blinded by a code number until all biochemical analyses were completed. ApoA1 standard (Sigma, UK) alongside human serum samples from patients and control subjects were diluted 1:1000 with phosphate buffered saline, pH 7.4 (PBS, Sigma, UK). ApoA1 standard and samples were then loaded onto ELISA Maxisorb plates (Nunc™, Denmark) and incubated for 1 hr. After washing with washing buffer (0.03% Tween 20 in PBS), the plates were blocked with 5% dried skimmed milk powder in PBS for 60 minutes. 100 μl ApoA1 primary antibody (rabbit) (CalBiochem, UK; 1:1000) was incubated in 96-well plates for 60 minutes. The plates were washed four times with wash buffer followed by the addition of 100 μl anti-rabbit secondary antibody (Cell Signalling, UK; 1:2000) to each well, and incubated for 60 minutes. All incubations were carried out on a shaker (600 rpm) at room temperature. Finally, after washing four times with wash buffer, 100 μl substrate (TMB One solution, Promega) was then added to each well and incubated at room temperature for 10 minutes. The plates were read with a plate reader (BIO-RAD, Model 680) at 450 nm. Statistical analysis of serum samples was performed by t-test using the Statistical Package for Social Scientists (SPSS/PC+; SPSS, Chicago). All measurements were replicated in an independent experiment.

TABLE 9
Summary of ApoA1 expressions in CSF, post-mortem
brain, liver tissues, RBC and sera in schizophrenia
patients and control subjects

| | | Sample size | |
|---|---|---|---|
| Tissue/body fluid | Technique | (patient/control) | Result* |
| CSF | SELDI-TOF | 41/40 | −35%; p = 0.00001 |
| Liver | 2D-DIGE | 15/15 | −30%; p = 0.017 |
| RBC | 2D-DIGE | 20/20 | −60%; p = 0.0034 |
| Serum | ELISA | 35/63 | −18%; p = 0.00039 |
| Brain | Western Blot | 8/8 | −32%; p = 0.07 |

*p value is derived from student t test.

### Example 4

Changes in ApoA1 (apolipoprotein A1) and TTR (transthyretin) proteins were initially identified in CSF from first-onset drug-naïve schizophrenia to patients. Both were found to be significantly down-regulated in CSF.

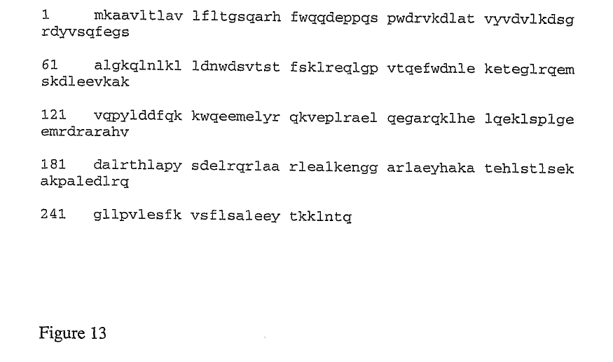

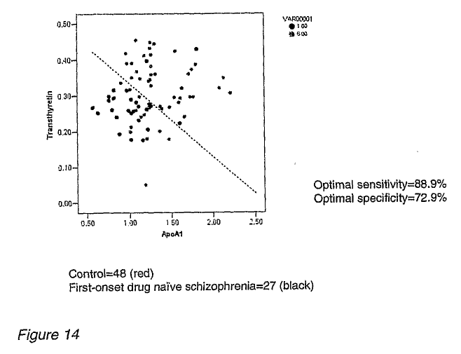

This Example investigated changes in ApoA1 and TTR in serum. Thus ELISA assays for both proteins were established and 27 schizophrenia (first-onset drug naïve) and 48 healthy volunteer sera were investigated. Both proteins were found to be significantly reduced in schizophrenia serum (apoA1: −18%; p=0.00039 and TTR: −15%; p=0.0007). See FIG. 13.

### REFERENCES

### All Incorporated Herein by Reference

1. Gottesman I I. Schizophrenia genesis: the origins of the madness. 1991:296.
2. Kringlen E. Twin studies in schizophrenia with special emphasis on concordance figures. Am J Med Genet 2000; 97(1):4-11.
3. Yolken R H, Torrey E F. Viruses, schizophrenia, and bipolar disorder. Clin Microbiol Rev 1995; 8(1):131-45.
4. Kosten T R, Ziedonis D M. Substance abuse and schizophrenia: editors' introduction. Schizophr Bull 1997; 23(2): 181-6.
5. Kendler K S. The season of birth of schizophrenic, neurotic and psychiatrically normal twins. Br J Psychiatry 1982; 141:186-90.
6. Reveley A M, Reveley M A, Clifford C A, Murray R M. Cerebral ventricular size in twins discordant for schizophrenia. Lancet 1982; 1 (8271):540-1.
7. Suddath R L, Christison G W, Torrey E F, Casanova M F, Weinberger D R. Anatomical abnormalities in the brains of monozygotic twins discordant for schizophrenia. N Engl J Med 1990; 322(12):789-94.
8. Iwamoto K, Bundo M, Kato T. Altered expression of mitochondria-related genes in postmortem brains of patients with bipolar disorder or schizophrenia, as revealed by large-scale DNA microarray analysis. Hum Mol Genet. 2005; 14(2):241-53.
9. Middleton F A, Mimics K, Pierri J N, Lewis D A, Levitt P. Gene expression profiling reveals alterations of specific metabolic pathways in schizophrenia. J Neurosci 2002; 22(7):2718-29.
10. Mimmack M L, Ryan M, Baba H, et al. Gene expression analysis in schizophrenia: reproducible up-regulation of several members of the apolipoprotein L family located in a high-susceptibility locus for schizophrenia on chromosome 22. Proc Natl Acad Sci U S A 2002; 99(7):4680-5.
11. Prabakaran S, Swatton J, Ryan M, et al. An integrative functional genomics approach reveals impaired brain energy metabolism in Schizophrenia. Mol. Psychiatry. 2004: 9(7):684-697, 643.
12. Haupt D W, Newcomer J W. Abnormalities in glucose regulation associated with mental illness and treatment. J Psychosom Res 2002; 53(4):925-33.
13. Heiskanen T, Niskanen L, Lyytikainen R, Saarinen P I, Hintikka J. Metabolic syndrome in patients with schizophrenia. J Clin Psychiatry 2003; 64(5):575-9;
14. Nicholson J K, Foxall P J, Spraul M, Farrant R D, Lindon J C. 750 MHz 1H and 1H-13C NMR spectroscopy of human blood plasma. Anal Chem 1995; 67(5):793-811.
15. Schreiber, G. The evolutionary and integrative roles of transthyretin in thyroid hormone homeostasis. J. Endocrinol 175, 61-73 (2002)
16. Reiber, H. Dynamics of brain-derived proteins in cerebrospinal fluid. Clin Chim Acta 310, 173-86 (2001).
17. Sullivan G M et al. Low levels of transthyretin in the CSF of depressed patients. Am Psychiatry 156:5 (1999)
18. Chen, M. L. & Chen, C. H. Comparative proteome analysis revealed up-regulation of transthyretin in rat brain under chronic clozapine treatment. J Psychiatr Res (2005).
19. Tang, N., Tornatore, P. & Weinberger, S. R. Current developments in SELDI affinity technology. Mass Spectrom Rev 23, 34-44 (2004).
20. Kemna, E. et al. Novel urine hepcidin assay by mass spectrometry. Blood (2005).
21. Swatton, J. E. et al. Increased MAP kinase activity in Alzheimer's and Down syndrome but not in schizophrenia human brain. Eur J Neurosci 19, 2711-9 (2004).
22. Ruetschi, U. et al. Identification of CSF biomarkers for frontotemporal dementia using SELDI-TOF. Exp Neurol (2005).
23. Schweigert, F. J., Wirth, K. & Raila, J. Characterization of the microheterogeneity of transthyretin in plasma and urine using SELDI-TOF-MS immunoassay. Proteome Sci 2, 5 (2004).
24. Morley, J. E. & Shafer, R. B. Thyroid function screening in new psychiatric admissions. Arch Intem Med 142, 591-3 (1982).
25. Ryan, W. G., Roddam, R. F. & Grizzle, W. E. Thyroid function screening in newly admitted psychiatric inpatients. Ann Clin Psychiatry 6, 7-12 (1994).
26. Kirkegaard, C. & Faber, J. The role of thyroid hormones in depression. Eur J Endocrinol 138, 1-9 (1998).
27. Hall, R. C. W., Stickney, S. & Beresford, T. P. Endocrine disease and behavior. Integr. Psychiatry 4, 122-135 (1986). End 35
28. Mooradian, A. D. et al (2004) Transciptional control pf apolipoprotein A-I gene expression in diabetes Diabetes 53, 513-520
29. Watts et al (2004) Diabetes 52, 803-811
30. Wen et al (2003), Acta Pharmacol Sin, 24 (10), 1001-1005
31. Middleton (2005), American Journal of Medical Genetics Part B, 136B, 12-25
32. Bossy-Wetzel, E., Schwarzenbacher, R., and Lipton, S. A. (2004) Nat Med 10 Suppl, S2-9.

1-75. (canceled)

76. A method selected from the group consisting of:
i) diagnosing or monitoring a psychotic disorder, or predisposition thereto, and
ii) monitoring efficacy of a therapy in a subject having, suspected of having, or suspected of being predisposed to, a psychotic disorder,
wherein said method comprises measuring the level of one or more biomarkers present in a sample taken from a subject, said biomarkers being selected from a transthyretin peptide comprising SEQ ID NO: 1 or a fragment thereof, an ApoA1 peptide comprising SEQ ID NO: 2 or a fragment thereof, VLDL, LDL and aromatic species.

77. A method of identifying an anti-psychotic substance, comprising measuring the level of one or more biomarkers present in a sample taken from a subject, said biomarkers being selected from a transthyretin peptide comprising SEQ ID NO: 1 or a fragment thereof an ApoA1 peptide comprising SEQ ID NO: 2 or a fragment thereof, VLDL, LDL and aromatic species.

78. The method according to claim 76, comprising comparing the levels of the one or more biomarkers in a sample taken from the subject with the level present in one or more samples taken from the subject prior to commencement of a therapy, and/or one or more samples taken from the subject at an earlier stage of a therapy.

79. The method according to claim 76, comprising detecting a change in the amount of the one or more biomarkers in samples taken on two or more occasions.

80. The method according to claim 76, comprising comparing the amount of the one or more biomarkers present in a sample with the level in one or more controls.

81. The method according to claim 80, wherein the controls are a normal control and/or a psychotic disorder control.

82. The method according to claim 76, wherein the level of one or more biomarkers is detected by one or more methods selected from NMR, SELDI (-TOF), MALDI (-TOF), a 1-D gel-based analysis, a 2-D gel-based analysis, mass spectrometry (MS), a LC-MS-based technique, direct or indirect, coupled or uncoupled enzymatic methods, electrochemical, spectrophotometric, fluorimetric, luminometric, spectrometric, polarimetric and chromatographic techniques, and immunological methods.

83. The method according to claim 76, wherein the biomarker is VLDL and/or LDL and the level thereof is detected by one or more methods selected from a liquid-phase chemical method, a physical method for separation of lipoproteins and an enzymatic assay.

84. The method according to claim 76, wherein the level of one or more biomarkers is detected using a sensor or biosensor comprising one or more enzymes, binding, receptor or transporter proteins, antibody, synthetic receptors or other selective binding molecules for direct or indirect detection of the biomarkers, said detection being coupled to an electrical, optical, acoustic, magnetic or thermal transducer.

85. The method according to claim 76, wherein the sample is selected from whole blood, blood serum, blood plasma, CSF, urine, saliva, or other bodily fluid, or breath, condensed breath or an extract or purification therefrom, or dilution thereof.

86. The method according to claim 76, wherein the subject is drug-naïve.

87. The method according to claim 76, wherein the psychotic disorder is a schizophrenic disorder.

88. The method according to claim 87, wherein the psychotic disorder is a bipolar disorder.

89. The method according to claim 76, further comprising a clinical or self-assessment of the subject.

90. The method according to claim 77, comprising comparing the levels of the one or more biomarkers in a sample taken from the subject with the level present in one or more samples taken from the subject prior to commencement of a therapy, and/or one or more samples taken from the subject at an earlier stage of a therapy.

91. The method according to claim 77, comprising detecting a change in the amount of the one or more biomarkers in samples taken on two or more occasions.

92. The method according to claim 77, comprising comparing the amount of the one or more biomarkers present in a sample with the level in one or more controls.

93. The method according to claim 92, wherein the controls are a normal control and/or a psychotic disorder control.

94. The method according to claim 77, wherein the level of one or more biomarkers is detected by one or more methods selected from NMR, SELDI (-TOF), MALDI (-TOF), a 1-D gel-based analysis, a 2-D gel-based analysis, mass spectrometry (MS), a LC-MS-based technique, direct or indirect, coupled or uncoupled enzymatic methods, electrochemical, spectrophotometric, fluorimetric, luminometric, spectrometric, polarimetric and chromatographic techniques, and immunological methods.

95. The method according to claim 77, wherein the biomarker is VLDL and/or LDL and the level thereof is detected by one or more methods selected from a liquid-phase chemical method, a physical method for separation of lipoproteins and an enzymatic assay.

96. The method according to claim 77, wherein the level of one or more biomarkers is detected using a sensor or biosensor comprising one or more enzymes, binding, receptor or transporter proteins, antibody, synthetic receptors or other selective binding molecules for direct or indirect detection of the biomarkers, said detection being coupled to an electrical, optical, acoustic, magnetic or thermal transducer.

97. The method according to claim 77, wherein the sample is selected from whole blood, blood serum, blood plasma, CSF, urine, saliva, or other bodily fluid, or breath, condensed breath or an extract or purification therefrom, or dilution thereof.

98. The method according to claim 77, wherein the subject is drug-naïve.

99. The method according to claim 77, wherein the psychotic disorder is a schizophrenic disorder.

100. The method according to claim 99, wherein the psychotic disorder is a bipolar disorder.

101. The method according to claim 77, further comprising a clinical or self-assessment of the subject.


SEQUENCE LISTING (2 sequences)

SEQ ID NO: 1 - protein, 117 aa, Homo sapiens
PLMVKVLDAVRGSPAINVAVHVFDKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 2 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ